(19) United States
(12) Patent Application Publication
(10) Pub. No.: US 2025/0314634 A1
(43) Pub. Date: Oct. 9, 2025

(54) OPTICAL SENSOR PATCH FOR THE MEASUREMENT OF WATER ACTIVITY

(71) Applicant: CORNELL UNIVERSITY, Ithaca, NY (US)

(72) Inventors: Abraham D. STROOCK, Ithaca, NY (US); I-Feng WU, Ithaca, NY (US); Piyush JAIN, Ithaca, NY (US); Sabyasachi SEN, Ithaca, NY (US); Annika E. HUBER, Cortland, NY (US)

(21) Appl. No.: 19/169,739

(22) Filed: Apr. 3, 2025

Related U.S. Application Data
(60) Provisional application No. 63/573,961, filed on Apr. 3, 2024.

(51) Int. Cl. G01N 33/24; A01G 25/16; G01N 21/64; G01N 21/84; G01N 33/00

(52) U.S. Cl. CPC G01N 33/246; G01N 21/6428; G01N 21/645; G01N 33/0098; A01G 25/167; G01N 2021/6432; G01N 2021/6439; G01N 2021/6484; G01N 2021/8466

(57) ABSTRACT

An optical sensor patch for measuring water activity is disclosed. The optical sensor patch includes a sheet configured to be in equilibrium with water activity in an environment of the optical sensor patch. A pair of distinct fluorescent dyes are dispersed within the sheet. The fluorescence spectra of the optical sensor patch is dependent on the water activity in the environment of the optical sensor patch. An optical implant and systems and methods for using the optical sensor patch are also disclosed.

This application claims the benefit of U.S. Provisional Patent Application Ser. No. 63/573,961, filed Apr. 3, 2024, which is hereby incorporated by reference in its entirety.

### GOVERNMENT FUNDING

This invention was made with government support under Grant Number FA9550-21-1-0283 awarded by the Air Force Office of Scientific Research and Grant Number DBI-2019674 awarded by the U.S. National Science Foundation. The government has certain rights in the invention.

### FIELD

The present technology relates to an optical sensor patch. More specifically, the present technology relates to an optical sensor patch for measuring water activity, as well as systems and methods of use thereof.

### BACKGROUND

Water is ubiquitous, important, and plays dominant roles in physical, chemical, and biological processes on earth. However, insufficient understanding toward water has left many unanswered questions, and hindered progress in various contexts. For example, the role of water in global climate change, the efficiency of water usage for agriculture, and the utilization of water in industry and energy management are not fully understood. Thus, further understanding of the availability of water in these contexts is urgent and essential.

Water activity (equivalently, water potential, relative humidity, fugacity, or the chemical potential of water) represents the thermodynamic availability of water to participate in physical, chemical, and biological contexts. For example, water activity plays a crucial role in food science on preservation for remaining the flavor and texture of the products. Water activity is also closely related to the osmolarity in mammalian cells, which delicately control the physiology of mammalian species. Water potential and relative humidity of the environment control the hydraulic status of plants, which is related to plant physiology and yield. The availability of water in soil is closely linked to geological topics and agriculture development. In synthetic topics, there are development of materials for energy management through the phase change of water. Also, water activity defines the design of synthetic porous membranes for separation and purification. However, robust and general technologies to capture water activity in various states and environments are lacking.

There are some existing technologies to capture water activities. For example, chilled mirror hygrometers provide high accuracy and rapid measurements, but all the measurements are ex situ and are high in cost. For liquid samples, a freezing point osmometer can offer measurements, but the accuracy is limited when the sample is close to pure water. Thermal couple psychrometers are widely used on measuring water activity in a wide range of contexts. However, a thermal couple psychrometer has complications due to transient signals, high temperature sensitivity, and higher costs for electronics. Tensiometers can capture the water status in soil, and MEMS tensiometers can measure both in and ex situ water activity. However, precise and complicated fabrication techniques are required for such devices.

This disclosure is directed to overcoming these and other deficiencies in the art.

### SUMMARY

One aspect of the present technology relates to an optical sensor patch for measuring water activity. The optical sensor patch includes a sheet configured to be in equilibrium with water activity in an environment of the optical sensor patch. A pair of distinct fluorescent dyes are dispersed within the sheet. The fluorescence spectra of the optical sensor patch is dependent on the water activity in the environment of the optical sensor patch.

Another aspect of the present technology relates to an optical implant. The optical implant includes a first window in a substrate configured to receive the optical sensor patch of the present technology. A first groove in the substrate is configured to receive a first optical fiber to direct light to and receive light from the optical sensor patch within the first window. A pointed tip is located proximate the first window. The pointed tip is configured for insertion of at least a portion of the optical implant into the environment for measuring water activity.

Another aspect of the present technology relates to a system for measuring water activity. The system includes the optical sensor patch of the present technology. A fiber optic cable is coupled to the optical sensor patch. A light source is coupled to the fiber optic cable to provide light directed at the optical sensor patch. A measurement device is coupled to the fiber optic cable to receive and analyze modulated light from the patch.

Yet another aspect of the present invention relates to a method of measuring water activity. The method includes providing the system including the optical sensor patch of the present technology. Changes in the fluorescence spectra of the optical sensor patch are measured. The water activity in the environment of the optical sensor patch is determined based on the changes in the fluorescence spectra.

The present technology advantageously provides an optical sensor patch that can be used to measure water activity in environments, such as in-vivo plant measurements. The optical sensor patch of the present technology can be used to form implants that are designed to be disposable (or reusable), minimally invasive, and capable of measuring various analytes/physiological parameters in plants over an extended period of time. The implants can be integrated with field-ready instrumentation, e.g. spectrometers and light sources, to provide a more easily fabricated, less complex sensor for measuring water activity in various environments.

### BRIEF DESCRIPTION OF THE DRAWINGS

FIG. 1 is a diagram of an exemplary system for using an optical sensor patch of the present technology in an optical implant for making in-planta measurements of water activity.

FIG. 2 is an illustration of an exemplary optical implant incorporating an optical sensor patch of the present technology.

FIG. 3A illustrates an optical sensor patch having two fluorescent dyes dispersed directly into a sheet.

FIG. 3B illustrates an optical sensor patch having two fluorescent dyes located in hydrogel nanoparticles dispersed in the sheet.

FIG. 4 is an image of another exemplary optical implant incorporating an optical sensor patch of the present technology.

FIG. 5 illustrates an exemplary method of forming an optical implant including the optical sensor patch of the present technology.

FIG. 6 is an illustration of another exemplary optical implant incorporating an optical sensor patch of the present technology.

FIG. 7 is a flowchart of an exemplary method of measuring water activity in an environment.

FIG. 8 is a diagram of an exemplary fabrication of an optical sensor patch of the present technology.

FIG. 9A is an exemplary setup for point probe measurements using an optical sensor patch of the present technology.

FIG. 9B is an exemplary setup for imaging of an optical sensor patch of the present technology.

FIG. 10 is a graph of exemplary spectra of fluorescent dyes for an exemplary optical sensor patch of the present technology.

FIGS. 11A-11D illustrate exemplary data for analysis of the spatial distribution of relative intensity for an optical sensor patch of the present technology. FIG. 11A is an image acquired at the OG emission channel with the excitation of OG excitation

$$( {F}_{{OG}_{ex}}^{{OG}_{em}} ) ;$$

FIG. 11B is an image acquired at the Rho emission channel with the excitation of OG excitation

$$( {F}_{{OG}_{ex}}^{{OG}_{em}} ) ;$$

FIG. 11C illustrates the calculated pixelwise relative intensity from the images from FIGS. 11A and 11B; and FIG. 11D is a pixel count distribution histogram plotted against pixel intensity.

FIGS. 12A is an exemplary optical sensor patch emission spectra in response to the decreasing water activity (from top curve to bottom curve: near saturation (a_{w}˜1) to undersaturation (a_{w}<1)).

FIG. 12B is a spectra decomposition example for point measurement analysis including: an emission spectrum; OG contribution; and Rho contribution.

FIG. 12C is a graph illustrating the characterization of relative intensity of the hydrogel nanoparticle based optical sensor patch against vapor and liquid, defined by NaCl solution) water potential.

FIG. 13A is a graph of the time elapse of relative intensities of the optical sensor patch in response to a step change of water potential in the vacuum chamber. The dashed line corresponds to fitted curve using exponential saturation function.

FIG. 13B is a schematic diagram of the temperature-controlled vacuum chamber.

FIG. 13C is a schematic model of optical sensor patch transient referring to the dashed box in FIG. 13B.

FIG. 14A is a schematic diagram of an optical cell for a soil drying experiment described in the examples herein.

FIG. 14B is an image of the optical cell with the dashed box indicating the optical sensor patch above the soil sample.

FIG. 14C is a graph of the time evolution of the spatiotemporal water potential distribution from top to bottom: t=5, 10, 15, 20, 25 hrs.

FIG. 15A illustrates a set of decomposed OG (emission peak: 520 nm) and Rho (emission peak: 568 nm) spectra corresponding the changing of water activity (corresponding to decreasing water activity).

FIG. 15B is a graph illustrating the characterization of relative intensity of dye-based optical sensor patch against vapor water potential.

FIG. 15C is a graph of the characterization of relative intensity of dye-based optical sensor patch against liquid water potential.

FIG. 16 is a graph of a background subtracted spectra of a dry signal, when the implant is exposed to ambient air, and a wet signal, where the implant is dipped in pure water. The spectrum changes due to the response of the hydrogel nanoparticles that are embedded in the optical sensor patch. The exposure time is 6 s.

FIG. 17 is a graph illustrating time lapse of a background-subtracted spectral response of the implant during a dry-down experiment of tomato. The time indicates the duration since the last watering of the plant, and the “air” plot shows the response of the implant before placing it into the stem. A 5-second integration time was used and the background signal of the implant was measured before placing it into the stem.

FIG. 18 shows AquaSheet as an optical sensing tool for water activity: (A) Contexts related to water activity (a_{w}): (i) Food science and preservation technologies. (ii) Biotechnology related to mammalian osmotic balance. (ii) Plant physiology of water-use. (iv) Soil science and hydrology. (v) Energy engineering. (vi) Membrane science for separations. (B) Schematic diagram of AquaSheet and its response to the water activity in its local environment: AquaDust nanogels (pink circles with pairs of conjugated dyes) are dispersed into the PDMS (gray rectangle) matrix. Upon swelling (blue arrow for increasing a_{w}) and shrinking (red arrow for decreasing a_{w}), the fluorescence emission from the nanogels shifts. (C) Schematic diagram of AquaSheet submerged in water vapor (left) and liquid solutions (right). For vapor case, vapor water molecules (light blue) diffuse through the PDMS matrix to reach equilibrium inside (a_{w,in}) and outside (a_{w,out}) the AquaSheet matrix. At equilibrium AquaDust reports the vapor water activity outside the AquaSheet. For liquid case, PDMS matrix serves as a barrier to exclude solutes from entry into the matrix such that AquaDust in AquaSheet responds to the changes in the liquid solution activity (a_{w,out}). (D) Photo of an AquaSheet sample. The red dashed rectangle indicates the zone containing AquaDust. (E) Measurement and response of

AquaSheet under different water activity: (Top) Spatially averaged measurements: (left) a representation of point probe system; (middle) a fluorescence emission spectrum of AquaSheet under near saturation (a_{w}˜1) with inset of swollen AquaDust in PDMS matrix; (right) a fluorescence emission spectrum of AquaSheet under unsaturation (0<a_{w}<1) with inset of shrunk AquaDust in PDMS matrix. (Bottom) Spatiotemporal measurements: (left) a representation of imaging system; (middle) a spatial distribution of calculated relative intensity (ζ) of AquaSheet under near saturation (a_{w}˜1) (right) a spatial distribution of calculated relative intensity (ζ) of AquaSheet under unsaturation (0<a_{w}<1).

FIG. 19 shows Fabrication of AquaSheet: (A) Schematic representation of synthesis of AquaDust nanogels: the polymerization through inverse microemulsion generates nanogels with diameters of 60˜120 nm; pairs of Alexa Fluor dyes are reacted to free amines in crosslinked gels; and a purified aqueous suspension is created by filtration and centrifugation. (B) Schematic presentation of the fabrication of AquaSheet: prepolymer and curing agent of PDMS are pre-mixed in the petri-dish. Prior to curing, the uncured polymer is oxidized in a low temperature air-plasma to form a hydrophilic surface layer, AquaDust solution is mixed manually with uncured PDMS to create emulsion-like mixture, and the AquaSheet is cured in oven at 50° C. for two days followed by 80° C. for one day.

FIG. 20 shows Reflectance probe measurements of spatial averaged response of AquaSheet to water activity. (A) A schematic diagram of reflectance probe system. (Detailed diagram is shown in FIG. S2). (B) Examples of the fluorescence emission spectra of AquaSheet at equilibrium with a series of undersaturated water vapor of activities imposed in a vacuum chamber (FIG. S1): a_{w}=0.65 (yellow) to a_{w}=0.99 (blue); the color bar (inset) shows corresponding water activity (a_{w}). For each water activity, three replicates were acquired; the plotted spectra refer to the median of the intensity of each wavelength among three replicates, and the shaded area refers to the upper and lower limit of each wavelength within three replicates. (C) Example of spectral decomposition of an emission spectrum (blue curve) into the contributions from both dyes (AF488 and AF568) using the ‘lsqnonneg’ function in MATLAB: x is the fractional ratio contribution by AF488, and the fractional emission of AF488 is plotted in green curve; y is the fractional ratio contribution by AF568, and the fractional emission of AF568 is plotted in yellow curve. The black dashed curve refers to the summation of the contribution from both dyes. (D) The characterization of relative intensity (ζ) of AquaSheet against vapor (blue, defined by vacuum chamber) and liquid (red, defined by NaCl solution¹) water activity.

FIG. 21 shows Transient of AquaSheet. (A) The time elapse (Δt=2 minutes between record points) of relative intensities (−) of AquaSheet in response to a step change of water activity in the vacuum chamber (a_{w,vc}). The red dashed line corresponds to fitted curve based on the exponential transient provided in Eq. (5). (B) A schematic diagram of experimental system. At t=0, a step change in the vapor pressure, Δp, was imposed in the vapor surrounding the AquaSheet sample, which corresponding to a step change in water activity,

$$Δ {a}_{w} = Δ p / {p}_{vap}^{sat} ( T ) \cdot {p}_{vap}^{sat} ( T )$$

is the saturation vapor pressure at temperature T. (C) This vapor pressure after step change imposes a new concentration of dissolved water within the AquaSheet matrix at its top boundary,c(x=L,t>0), which can be related by Henry's law: c(x=L,t>0)=k_{H}pᵥₐₚ, where k_{H} [Pa/M] is the Henry's constant of water molecules dissolving in PDMS matrix. We modeled the transient progression of the concentration in the matrix (c(x,t)) with the one-dimensional, time-dependent diffusion equation, with diffusivity, D [m²/s]. This diagram refers to the red dashed box in (B).

FIG. 22 shows AquaSheet reporting spatiotemporal distribution of water activity (potential) of drying soil: (A) Schematic diagrams showing top view (top) and side view (bottom) of the optical cell for soil drying experiment. With this design, the top and bottom of the soil sample are blocked by the plexiglass and the cell. The other three sides are sealed with an O-ring such that the soil sample is only exposed on one side to the ambient air; this geometry aimed to create a one-dimensional progression of drying through the soil sample. (B) Photo of the optical cell. The red dashed box indicates the zone of AquaSheet that contains AquaDust and from which measurements were captured. (C) The time evolution of the spatial distribution of water activity (potential) in the soil during drying. This experiment was performed as follows: The dry soil sample was mixed with water in a plastic container (˜20 ml) with a spatula. The wet soil sample was then packed into the optical cell until the top surface was flat. AquaSheet was then placed between the soil and upper plexiglass lid. The measurement was performed on the benchtop with a fan directed at the exposed surface of the soil to minimize the boundary layer resistance to vapor transfer. The relative humidity of ambient is around 50% (i.e., a_{w}=0.5). For this experiment, the imaging system described in Section Theory and Design was employed to acquire images across 45 hours. (Top to bottom: t=0, 5, 15, 25, 35 and 45 hours.) The white pixels presented in water activity distribution refer to the saturated pixels of the raw images.

FIG. 23 shows the schematic diagram of the vacuum chamber The water source was submerged in a constant-temperature water bath (T=18° C.). A vacuum pump extracted vapor water from the water source, passing it through the vacuum chamber, which was connected to a circulated constant-temperature water bath (T=15° C.). The temperature of the water source was intentionally set higher than that of the chamber circulation to ensure a sufficient vapor source and prevent condensation in the upstream pipeline. Vapor pressure, p_{vap,VC} [Pa] within the chamber was regulated using two needle valves, positioned upstream and downstream of the chamber, and p_{vap,VC} was measured by a capacitance pressure gauge. The AquaSheet sample (pink rectangle) was positioned on the temperature-controlled floor of the chamber (temperature, T_{VC} [° C.]), monitored in real time by a resistance temperature detector (RTD). The water activity imposed at the surface of the sample is

$${a}_{w , VC} = {p}_{vap , VC} / {p}_{vap}^{sat} ( {T}_{VC} ) ,$$

as be calculated through Eq (1).

FIG. 24 shows the schematic diagram of the instrumentation for reflectance probe measurements: A mercury lamp served as the light source, emitting light across various wavelengths. The emitted light passed through a band-pass filter (465˜505 nm, labeled as “Ex filter”) to select specific excitation wavelengths. Subsequently, the excitation light was transmitted through a six-fiber bundle, evenly illuminating the AquaSheet sample. The emission light, collected through a single central fiber within the same bundle, underwent further filtration using a high-pass filter (>510 nm, marked as “Em filter”) to minimize excitation light bleed-through. The collected light was then directed to a spectrometer and recorded by Ocean View software, with an integration time ranging from 1 to 4 seconds. This integration time was adjusted to achieve the desired signal intensity, typically exceeding 5,000 counts to ensure a robust signal-to-noise ratio while avoiding detector saturation, which occurs at counts exceeding 60,000.

FIG. 25 shows the schematic diagram of the instrumentation for imaging measurements: The imaging system comprised three main components: An LED light source, CCD camera with lens, and the Arduino-driven automatic filter wheel. A high-power light guide coupled LED source with a customized coupler served as the light source. Excitation light was directed through a band-pass filter (470˜500 nm) to select the desired excitation wavelength. Subsequently, the excitation light passed through a ring light to ensure uniform illumination of the sample. Emission images were captured using an FLIR CCD camera, which was connected to the Arduino-driven automatic filter wheel. This filter wheel, positioned between the sample and the CCD camera and labeled as the “Emission filter wheel,” housed two band-pass filters: one for the AF488 emission channel (515˜535 nm) and another for the AF568 emission channel (574˜596 nm). The filter wheel automatically shifted to the appropriate position for the acquisition of either AF488 or AF568 emission channels. The LED light source, the emission filter wheel, and the CCD camera were connected to the LabView software for images acquisition. Each replicate involved the acquisition of two images: the first image

$$( {I}_{AF 48 {8}_{ex}}^{AF 48 {8}_{em}} )$$

was captured with AF488 excitation band and detected at AF488 emission band (channel 1); the second image

$$( {I}_{AF 48 {8}_{ex}}^{AF 56 {8}_{em}} )$$

was captured by being excited at AF488 excitation band and collected at AF568 emission band (channel 2).

FIG. 26 shows the spectra of AF488 and AF568 dyes provided by the chemical vendor: The dashed green line represents the excitation of AF488, and the solid green line represents the emission of AF488. The dashed yellow line represents the excitation of AF568, and the solid yellow line represents the emission of AF568. The two vertical dashed black lines confine the AF488 excitation channel defined by the bandpass filter (470˜500 nm). The first and second vertical solid black lines form the left confines the AF488 emission channel (channel 1) defined by the bandpass filter (515˜535 nm). The third and fourth vertical solid black lines form the left confines the AF568 emission channel (channel 2) defined by the bandpass filter (574˜596 nm). The blue shaded area stands for the AF488 emission at the AF488 emission channel; the red shaded area stands for the AF488 emission at the AF568 emission channel.

FIG. 27 shows An example of analysis and determination for ζ_{corr} based on the pixel-wise relative intensity (ζ_{corr,i}) from ratiometric imaging measurements: This is a demonstration of determination of the relative intensity ζ_{corr} under a steady water activity a_{w} based on the ratiometric imaging measurements. (A) The first image

$$( {I}_{AF 48 {8}_{ex}}^{AF 48 {8}_{em}} )$$

was captured with AF488 excitation band and detected at AF488 emission band (channel 1), and the red dashed area is referred to the region of interest for analysis. (B) The image

$$( {I}_{AF 48 {8}_{ex}}^{AF 56 {8}_{em}} )$$

was captured by being excited at AF488 excitation band and collected at AF568 emission band (channel 2), and the red dashed area is referred to the region of interest for analysis (same area in (A)). (C) The spatial distribution of calculated ζ_{corr,i} using Eq. (5) and images from (A) and (B). The black area refers to the pristine PDMS without any AquaDust. (D) The pixel count distribution plotted against relative intensity. The vertical red line indicated the median of pixel-wise relative intensity ζ_{corr,i}, which refers to the relative intensity ζ_{corr}(a_{w}) under this known, steady water activity a_{w}.

FIG. 28 shows the calibration of AquaSheet through ratiometric imaging method: (A) The raw images of channel 1

$$( {I}_{AF 48 {8}_{ex}}^{AF 48 {8}_{em}} )$$

and channel 2

$$( {I}_{AF 48 {8}_{ex}}^{AF 56 {8}_{em}} ) .$$

The images at the top row are AquaSheet at steady, near saturation water activity (a_{w}=0.985±0.005) and the images at the lower row are AquaShee at steady, undersaturation water activity (a_{w}=0.537±0.005). The rectangle in the middle of every image is the AquaSheet with AquaDust embedded in PDMS matrix, and this refers to the region of interest for the drying analysis. The reflectance at the margin originates from the lateral transmittance of the fluorescence in pristine PDMS, which is not considered to be the fluorescence of AquaSheet. (B) The calibration curve of AquaSheet across water activity ranging from 0.985±0.005 to 0.537±0.005. At each on-set upstream valve opening, three replicates were taken, and the real-time water activities were recorded. The errors presented here are the standard deviation of the pixelwise relative intensity, ζ_{corr}, with respect to median of the pixelwise distribution, ζ_{corr}(a_{w}).

FIG. 29 shows the theoretical evaluation of hydraulic properties of soil and AquaSheet: (A) The schematic diagram of AquaSheet on top of the soil, which refers to the region of interest as shown in FIG. 5 (A-B). The x axis is defined along the drying direction, and the soil is open to ambient at x=0. The y axis is defined as the axis which is perpendicular to the drying direction. The z axis is the through-thickness axis of the region of interest. The sample is with the length lₓ along x axis, width l_{y} along y axis, thickness of AquaSheet hₚ and the thickness of soil hₛ. (B) The circuit diagram refers to the possible hydraulic pathway in the whole system shown in (A). The hydraulic resistances, Rₚ, Rₗ and Rᵥ are the reciprocal of the hydraulic conductances for AquaSheet (Kₚ), liquid in soil (Kₗ) and vapor in soil (Kᵥ), respectively. The resistances are in parallel, which indicates that water mass flow will pass through the path with highest conductance. (C) The theoretical value of liquid conductance in soil (Kₗ, blue solid line), vapor conductance in soil (Kᵥ, red solid line), conductance in AquaSheet based on Harley, 2012 (K_{p,Harley}, green shaded area) and conductance in AquaSheet based on Barrie, 1969 (K_{p,Barries}, cyan shaded area) against water potential. The red dashed line refers to the air entry potential (1/a). (D) The theoretical value of liquid capacitance in soil (Cₗ, blue solid line), vapor capacitance in soil (Cᵥ, red solid line), capacitance in AquaSheet based on Harley, 2012 (C_{p,Harley}, green dashed line) and capacitance in AquaSheet based on Barrie, 1969 (C_{p,Barries}, cyan dashed line) against water potential. The red dashed line refers to the air entry potential (1/a).

FIG. 30 shows the signal and background intensities comparison for (A) calibration and (B) soil drying experiment with ratiometric imaging method: (A) The median of the pixel intensity of AquaSheet (blue) and background (red) for both channel 1 and channel 2 under a steady water activity during calibration process. In this case, the background represents a pristine PDMS on top of a silicon wafer. The left part of the figure shows the signal and background intensities at high water activity (a_{w}=0.985±0.005), and the right part of the figure shows the signal and background intensities at low water activity (a_{w}=0.537±0.005). The error bars indicate the standard deviation of the pixel intensity distribution with respect to the median value. (B) The median of the pixel intensity of AquaSheet (blue) and background (red) for both channel 1 and channel 2 during soil drying process. In this case, the background represents a pristine PDMS on top of soil. The left part of the figure shows the signal and background intensities after 5 hours of drying (the second panel shown in FIG. 5(C)), and the right part of the figure shows the signal and background intensities after 45 hours of drying (the sixth panel shown in FIG. 5(C)). The error bars indicate the standard deviation of the pixel intensity distribution with respect to the median value.

FIG. 31 shows one dimensional drying profile of soil: This is the one-dimensional drying profile of the two-dimensional spatiotemporal distribution of water potential shown in FIG. 5(C). The median of water activity of each x coordinate was extracted to plot the progression of drying process in solid line across 0˜50 hours. The shaded area represents the standard deviation with respect to the medians.

FIG. 32 shows the spatiotemporal distribution of water activity (potential) of drying soil with background subtraction: (A) The time evolution of the spatial distribution of water activity (potential) in the soil during drying with background subtraction during analysis. (B) One dimensional drying profile of soil extracted from

(A). The solid lines refer to the median of all y pixels at each x pixel

### DETAILED DESCRIPTION

The present technology relates to an optical sensor patch. More specifically, the present technology relates to an optical sensor patch for measuring water activity, as well as systems and methods of use thereof.

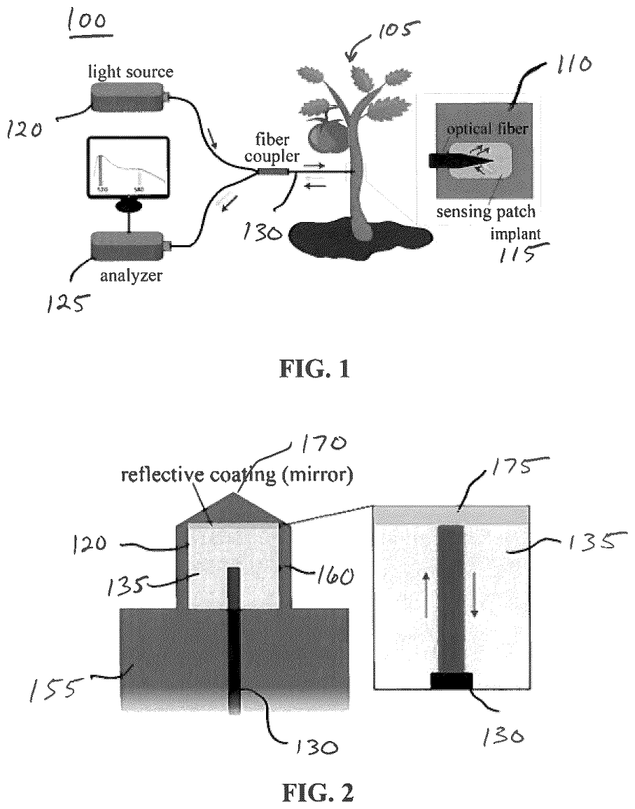

FIG. 1 illustrates a system 100 for measuring water activity in an environment 105 using an optical sensor patch 110. In this example, the environment 105 is an in vivo plant. It is to be understood that the system 100 can be utilized to measure water activity in any environment where such measurements are desired. Water activity (equivalently, water potential, relative humidity, fugacity, or the chemical potential of water) represents the thermodynamic availability of water to participate in physical, chemical, and biological contexts. The present system can be used to measure water activity for any application known in the art. Although the measurement of water activity is described herein, it is to be understood that optical sensor patch 110 can be configured to respond to specific analytes, stimuli, or status of the environment 105 (e.g., a plant) in accordance with the methods disclosed herein.

The system 100 includes the optical sensor patch 110 located in an optical implant 115, a light source 120, analyzer 125, and fiber optic cable 130, although the system 100 could include other types and/or numbers of elements or devices in other combinations. The system 100 provides an optical sensor patch that that is easy to manufacture and can be used to form a disposable (or reusable) and minimally invasive implant capable of measuring various analytes/physiological parameters, including water activity, in various environments. The system 100 further utilizes field-ready instrumentation, e.g. spectrometers and light sources, to a less complex system for measuring water activity in various environments.

Referring now to FIGS. 1 and 2, the system 100 includes optical sensor patch 110 incorporated in optical implant 115, which is shown in further detail in FIG. 2. The optical sensor patch 110, as described herein, can be designed to react to diverse physiological parameters in the environment 105, such as a plant, thereby causing corresponding changes in its optical properties. The optical sensor patch 110 includes a sheet 135 configured to be in equilibrium with water activity in the environment 105, such as the in vivo plant shown in FIG. 1, of the optical sensor patch 110. In one example, the sheet 135 is a polydimethylsiloxane (PDMS) matrix that can be formed to encapsulate materials therein, although the sheet 160 may be formed of other suitable materials.

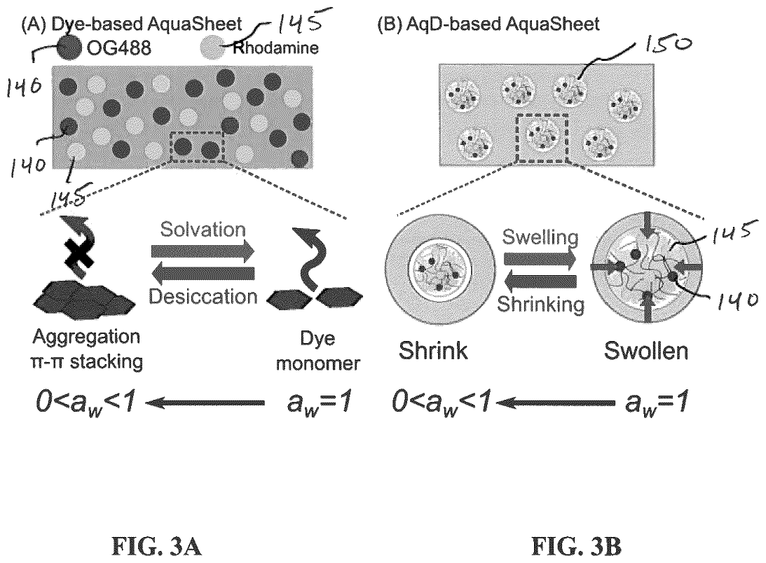

Referring now to FIGS. 3A and 3B, optical sensor patch 110 includes a pair of distinct fluorescent dyes 140 and 145 dispersed within the sheet 135, such as a PDMS matrix. In one example, the pair of fluorescent dyes are Oregon Green 488 (2′,7′-Difluorofluorescein, OG) and Rhodamine B (Rho) manufactured by Thermo Fisher Scientific, although other fluorescent dyes may be employed. As described herein, the pair of fluorescent dyes 140 and 145 are configured to provide a fluorescence spectra for the optical sensor patch 110 that is dependent on the water activity in the environment 105 of the optical sensor patch 110. The relative intensity of fluorescence can be measured using spectra deconvolution, as described herein. The acquired spectra are composed of the relative contribution of the emissions of both dyes 140 and 145.

In one example, as shown in FIG. 3A, the pair of fluorescent dyes 140 and 145 are dispersed directly into the matrix of sheet 135 during fabrication, as described below. In this example, changes in the fluorescence spectra of the optical sensor patch 110 are based on changes in self-quenching of the pair of distinct fluorescent dyes 140 and 145.

In this example, the self-quenching behavior of fluorescent dyes refers to the decrease of fluorescence emission due to the physical or chemical interaction between dye molecules and other molecules. One type of quenching can be attributed to the conjugation of metal complex to the other certain quencher, such as Ru (II) complex being quenched by oxygen molecules, as described in Castellano, F.N., et al., “A Water-Soluble Luminescence Oxygen Sensor.” Photochemistry and Photobiology, 67(2): 179-183 (2008), and Borisov, S.M., et al., “Optical biosensors.” Chem Rev, 108(2): 423-61 (2008), the disclosures of which are incorporated by reference herein in their entirety. The other type of quenching can be attributed to the aggregation of fluorescent dyes due to strong π-π stacking of dye molecules, which is called aggregation-caused quenching (ACQ), as disclosed in Zalmi, G.A., et al., “Recent Advances in Aggregation-Induced Emission Active Materials for Sensing of Biologically Important Molecules and Drug Delivery System.” Molecules, 27(1) (2021); Zhang, J., S., et al., “Aggregation-Induced Intersystem Crossing: Rational Design for Phosphorescence Manipulation.” J Phys Chem B, 124(11): 2238-2244 (2020); and Zhao, Z., et al., “Aggregation-Induced Emission: New Vistas at the Aggregate Level.” Angew Chem Int Ed Engl, 59(25): 9888-9907 (2020), the disclosures of which are incorporated by reference herein in their entirety. The explanation of ACQ can be linked to the exciton theory as disclosed in Kasha, M., et al., “The exciton model in molecular spectroscopy.” Pure and Applied Chemistry, 11(3-4): 371-392 (1965), the disclosure of which is incorporated by reference herein in its entirety, which provided the explanation of the relation between dye molecules aggregations and resulting luminescence (including fluorescence and phosphorescence).

A practical example of ACQ is that when the dyes are in diluted conditions, the dye molecules can emit fluorescence; however, in the more condensed conditions, the formation of non-emissive aggregation will strongly undermine the fluorescence, as disclosed in Chen, G., et al., “Conjugation-Induced Rigidity in Twisting Molecules: Filling the Gap Between Aggregation-Caused Quenching and Aggregation-Induced Emission.” Adv Mater, 27(30): 4496-4501 (2015) and Andreiuk, B., et al., “Fighting Aggregation-Caused Quenching and Leakage of Dyes in Fluorescent Polymer Nanoparticles: Universal Role of Counterion.” Chem Asian J, 14(6): 836-846 (2019), the disclosures of which are incorporated by reference herein in their entirety. The Jablonski diagram has been employed to explain that π-π stacking of aromatic rings on dye molecules enhance intersystem crossing and lead to the shift of fluorescence to phosphorescence, leading to lower fluorescence, as disclosed in Zhang, J., S., et al., “Aggregation-Induced Intersystem Crossing: Rational Design for Phosphorescence Manipulation.” J Phys Chem B, 124(11): 2238-2244 (2020); Zhao, Z., et al., “Aggregation-Induced Emission: New Vistas at the Aggregate Level.” Angew Chem Int Ed Engl, 59(25): 9888-9907 (2020); and Jablonski, A., “Efficiency of Anti-Stokes Fluorescence in Dyes.” Nature, 131(3319): 839-840 (1933), the disclosures of which are incorporated by reference herein in their entirety.

As shown in FIG. 3A, when the environment 105 is under desiccation (decreasing water activity), the dye concentration becomes higher and leads to lower fluorescent emission. On the other hand, when the environment 105 is under solvation, the dye concentration becomes diluted and leads to higher fluorescent emission.

In another example, as shown in FIG. 3B, the pair of fluorescent dyes 140 and 145 are covalently linked in a polymer matrix of a hydrogel nanoparticles 150 dispersed in the sheet 135. In this example, changes in the fluorescence spectra of the optical sensor patch 110 are based on changes in self-quenching of the pair of distinct fluorescent dyes 140 and 145 and changes in Förster Resonance Energy Transfer (FRET) between the pair of distinct fluorescent dyes 140 and 145.

The hydrogel nanoparticles 150 measure water activities through the swelling and shrinking of the hydrogel matrix. When the hydrogel nanoparticles 150 experience increasing water activity, the intermolecular distance between the dyes 140 and 145 will increase, leading to the decrease of Förster resonance energy transfer (FRET) efficiency. On the other hand, decreasing water activity will decrease the intermolecular distance between the dyes 140 and 145, leading to an increase of FRET efficiency as depicted in FIG. 3B. However, when the hydrogel nanoparticles 140 are encapsulated in the PDMS matrix of sheet 135, the fluorescent emission can be attributed to the combination of both FRET behavior and the dye quenching phenomena.

Referring again to FIG. 2, the optical sensor patch 110 is shown in optical implant 115, although as described herein optical sensor patch 110 can be employed in other types of optical implants. In this example, optical implant 115 includes a substrate 155 that is formed of polymethyl methacrylate (PMMA), although other transparent plastic materials may be employed for substrate 155. In one example, the substrate 155 has a thickness of about 0.8 mm, although other thicknesses can be employed.

The substrate 155 includes a window 160 configured to receive the optical sensor patch 110 therein. The substrate 155 further includes a groove 165 located therein to receive the optical fiber 130 to direct light to and receive light from the optical sensor patch 110 within the window 160 during operation of optical implant 115. In this example, substrate 155 further includes a pointed tip 170 located proximate the window 160. The pointed tip 170 configured for insertion of at least a portion of the optical implant 115 into an environment 105, such as an in vivo plant. Accordingly, the optical implant 115 can be implanted, by way of example, in a plant to perform in vivo measurements of changes in the fluorescence spectra of the optic sensor patch 110 based on changes in the water activity in the in vivo environment of the plant. In this example, the substrate 155 further includes a reflective coating 175 located in window 160 to enhance light reflection to improve signal intensity for the collected fluorescence emitted from the optical sensor patch 110. In one example, reflective coating 175 is sprayed onto the substrate 155 with a paint that contains metallic particles (e.g., Krylon, Looking glass paint). In another example, the reflective coating 175 is applied using evaporation to deposit metals (e.g., gold, silver).

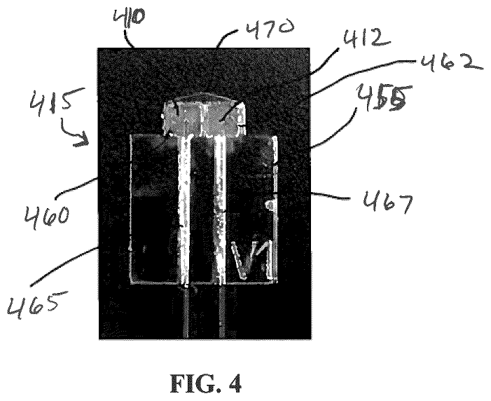

Referring now to FIG. 4, another exemplary optical implant 415 including optical sensor patch 410 is shown. In this example, optical sensor patch 410 is the same as optical sensor patch 110 described above. In this example, optical implant 415 includes a substrate 455 that is formed of polymethyl methacrylate (PMMA), although other transparent plastic materials may be employed for substrate 455. In one example, the substrate 455 has a thickness of about 0.8 mm, although other thicknesses can be employed.

The substrate 455 includes a first window 460 configured to receive the optical sensor patch 410 therein, as well as a second window 462 configured to receive a second optical sensor patch 412 therein. The substrate 455 further includes a first groove 465 located therein to receive an optical fiber to direct light to and receive light from the optical sensor patch 410 within the window 460 during operation of optical implant 415, and a second groove 467 located therein to receive an additional optical fiber to direct light to and receive light from the optical sensor patch 412 within the window 462 during operation of optical implant 415. In this example, substrate 455 further includes a pointed tip 470 located proximate the first and second windows 460 and 462. The substrate 455 may also include a reflective coating as described above. In one example, the second optical sensor patch 412 serves as a reference for the fluorescence spectra from the optical sensor patch 410.

FIG. 5 illustrates an exemplary method of fabricating the optical implant 115, as shown in FIG. 2. The exemplary method can also be used in the same manner to form optical implant 515 as shown in FIG. 4. First, the substrate 155, formed of PMMA (thickness 0.8 mm) in this example, is cut using a CO₂ laser cutter. Window 160 is defined to provide an area for optical sensor patch 110 and groove 165 is formed to securely fit an optical fiber, such as optical fiber 130 shown in FIG. 1. The pointed tip 170 of optical implant 115 allows for easy insertion, by way of example only, into plants. Next, an optical fiber, such as a 200 μm silica fiber, is inserted into groove 165 of optical implant 115. The mixture for the optical sensor patch 110, as described in further detail below, is then poured into window 160. The optical sensor patch 110 is then cured in a controlled environment with fixed temperature and humidity, such that the optical fiber is embedded in sheet 135 of optical sensor patch 110 to direct and receive light therefrom. The optical implant 555 shown in FIG. 5 is fabricated in the same manner. It is to be understand that optical implant 115 (FIG. 2) or optical implant 555 (FIG. 5) can be fabricated in other manners.

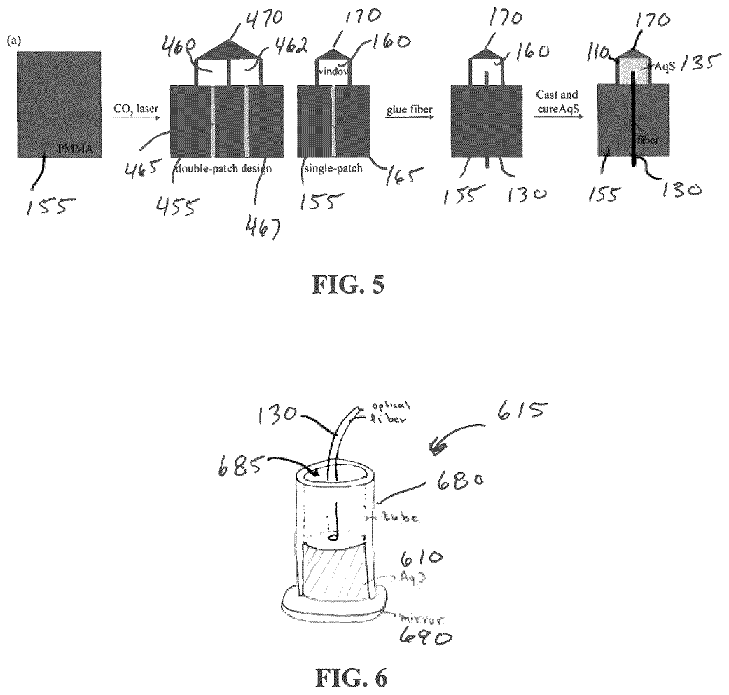

FIG. 6 illustrates another exemplary optical implant 615 including an optical sensor patch 610. In this example, the optical sensor patch 610 is embedded in a hollow tube 680 that has an opening 685 that exposes the optical patch 610 to the environment, such as environment 105 shown in FIG. 1. The fiber, such as fiber 130 shown in FIG. 2, is also inserted into the tube 680 and sits on top of the optical sensor patch 610. To enhance signal intensity, the tube 680 can equipped with a mirror 690 at its surface opposite the opening 685. Optical implant 615 could be used, for example, as a dipping probe for measuring water potential of different solutions. It is to be understood that other configurations of optical implants can be employed.

Referring again to FIG. 1, system 100 further includes light source 120 that is connected to fiber optic cable 130, such as a through a coupler and a reusable connector, to deliver light to the optical sensing patch 110 of the optical implant 115. This configuration enables the end of the optical fiber 130 not located in the optical implant 115 to remain outside the plant and be connected to the instrumentation via the connector during measurements.

Light source can be any suitable light source for exciting fluorescence in the optical sensor patch 110. System 100 further includes measurement device 125, such as a spectrometer or camera to receive and capture light emitted from the optical sensor patch 110. The system 100 may further include a computing device having a memory and one or more processing devices configured to further analyze the spectra obtained by the measurement device 125 using known methods. Optical fiber 130 can be a 200 μm optical, although other sizes of optical fiber can be employed.

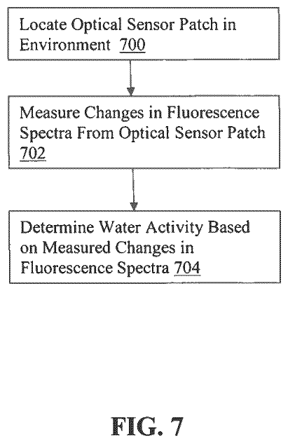

FIG. 7 is a flowchart of an exemplary method of measuring water activity in an environment, such as the environment 105 shown in FIG. 1, using system 100. In step 700, optical sensor patch 110, 410, 610 is located in the environment. In one example, optical implant 115 (FIG. 2) or optical implant 415 (FIG. 4) is utilized to implant the optical sensor patch 110, for example, into an in vivo plant. In another example, optical implant 615 is used to dispose the optical sensor patch 610 in a solution, by way of example only.

Next, in step 702, changes in the fluorescence spectra of the optical sensor patch 110, 410, 610 based on light directed to optical sensor patch 110, 410, 610 from light source 120 through optical fiber 130 are measured using the measurement device 125. The fluorescence spectra reacts to changes in water activity as described above with respect to FIGS. 3A and 3B.

In step 704, the water activity in the environment of the optical sensor patch 110, 410, 610 is determined based on the changes in the fluorescence spectra. Water activity is correlated to the changes in spectra as described in the examples herein. The water activity can be correlated with one or more analytes or physical parameters of the plant over a period of time.

### EXAMPLES

Examples of composite optical sensor patches were developed to perform point and 2D spatial water activity measurements in complex environments. Two different fluorescent dyes were dispersed in a precursor of a silicone elastomer before crosslinking to form thin sheets of material with fluorescence spectra that depend on the activity of water, a_{w}, with which it is at equilibrium. Two different methods were employed for the incorporation of the dyes within the silicone matrix.

### Example 1—Optical Sensor Patch Formed With Free Dyes

In another example, free dyes (in aqueous solution) were dispersed into the silicone matrix. The spectral changes in fluorescence of the optical sensor patch are interpreted as a function of water activity due to changes in the self-quenching of the dyes with changes in their hydration state. This design utilizes the dye pairs directly, resulting in a less complicated and lower cost alternative.

### Example 1—Materials and Methods

Oregon Green 488 (2′,7′-Difluorofluorescein, OG) and Rhodamine B (Rho) were purchased from Thermo Fisher Scientific. Polydimethylsiloxane (PDMS) precursor and curing agent kit (Sylgard 184) was purchased from Dow Corning. Deionized water (DI water, resistivity=18.2 MΩ·cm @25° C., Milli-Q Merck); N, N-Dimethyl formamide (DMF, Anhydrous) was purchased from Mallinckrodt Inc.

To prepare the dye solution, Rho solid powder was dissolved in deionized water and vortex for 1 minute to formulate Rho aqueous solution with a concentration of 0.01 mg Rho/1 ml DI water. OG solid powder was dissolved in DMF and vortex for 1 minute to formulate OG/DMF solution with a concentration of 1 mg Rho/1 ml DMF. Then, 2.5 μl of OG/DMF solution was pipetted and added to 25 μl Rho aqueous solution and followed by vortex for 1 minute to formulate dye solution.

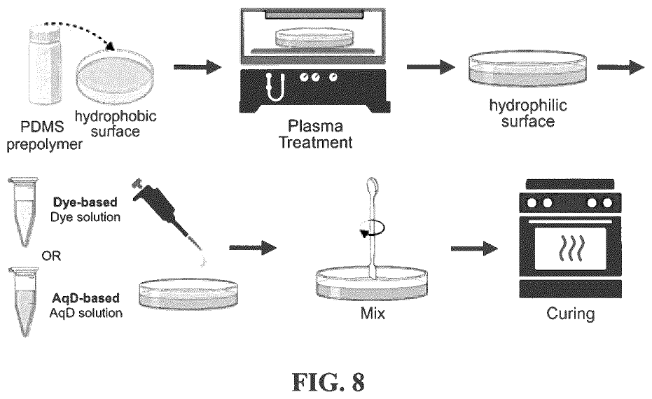

The optical sensor patch was fabricated as shown in FIG. 8. First, 0.25 g of PDMS precursor was measured in a petri-dish (35 mm in diameter, Corning). Five minutes wait time was employed to level the precursor by gravity for largest surface area. Then, the surface of PDMS precursor was oxidized with air plasma (Plasma Cleaner, Harrick) for 4 minutes to enhance the hydrophilicity, as described in McDonald, J.C., et al., “Fabrication of microfluidic systems in poly (dimethylsiloxane).” ELECTROPHORESIS: An International Journal, 21(1): 27-40 (2000) and Ge, M., et al., “A “PDMS-in-water” emulsion enables mechanochemically robust superhydrophobic surfaces with self-healing nature.” Nanoscale Horizons, 5(1): 65-73 (2020), the disclosures of which are incorporated herein by reference in their entirety.

The total 27.5 μl dye solution was pipetted onto the oxidized PDMS precursor surface immediately after moving the precursor out of the plasma chamber, followed by mixing manually with spatulas until the mixture became fully cloudy with no obvious dye solution floating on the surface. Next, 0.025 g of PDMS curing agent was added into the dye/precursor mixture (based on the ratio of 10:1 precursor to curing agent ratio) and mixed manually again for 5 minutes. The mixture was then degassed in a vacuum desiccator with a vacuum pump (RV12, Edwards) for 20 minutes. To define the thickness of the optical sensor patch, a pristine PDMS (with same precursor to curing ratio as 10:1) with the same thickness was fabricated, and a well-defined circular mold was cut with the AcuPonch Biopsy Punch (5 mm in diameter, Acuderm Inc.). The PDMS mold was placed onto a new petri-dish (100 mm in diameter, Falcon), and the degassed uncured optical sensor patch was cast into the well until the meniscus reached the same level as PDMS mold. The uncured optical sensor patch was cured under 50° C. for two days avoiding the formation of bubbles based on rapid water evaporation and followed by 80° C. for one day to cure the optical sensor patch thoroughly.

### Example 2—Optical Sensor Patch Formed With Hydrogel Nanoparticles

In an example, pre-formed hydrogel nanoparticles in which the pairs of dyes are covalently linked to the polymer matrix were dispersed in a silicone substrate. The particles have fluorescence response to changes in water activity as described in Jain, P., et al., “A Minimally Disruptive Method for Measuring Water Potential in Planta using Hydrogel Nanoreporters,” Proc Natl Acad Sci USA, 118(23) (2021) and U.S. Pat. No. 11,536,660 to Stroock, A., et al., the disclosures of which are incorporated herein by reference in their entirety. In this formulation, the spectral changes of the optical sensor patch fluorescence are interpreted as a function of water activity due to a combination of changes in self-quenching and changes in Förster Resonance Energy Transfer between the pairs of dyes with changes in the hydration state of the system.

### Materials and Methods

Acrylamide (AAm) (40% (w/v)), N,N-methylene bisacrylamide (BisAAm, >98%), Ammonium Persulfate (APS, >99.99%), Dichlorodimethylsilane (DCMS, >99%) were purchased from Sigma-Aldrich; Tetramethylethylenediamine (TEMED, Electrophoresis grade) was purchased from Fisher Scientific; N-aminopropyl methacrylamide (APMA, >98%) was purchased from Polysciences Inc; Dioctyl Sulfoccinate Sodium salt (AOT, 96%) and Polyoxyethylene(4)lauryl ether(Brij30) were purchased from ACROS Organics; n-Hexane (95%, HPLC Grade) was purchased from Millipore Sigma; N,N-Dimethyl formamide (DMF, Anhydrous) was purchased from Mallinckrodt Inc.; Oregon Green 488 N-hydroxysuccinimidyl ester (OG-NHS) and N-hydroxy succinimidyl ester Rhodamine (RH-NHS), were purchased from Thermo Fisher Scientific; Ethanol (Anhydrous, 100%) and Isopropyl alcohol (IPA) (99%) were purchased from VWR International; Phosphate-buffered saline (PBS) 1× tablet (10 mM Phosphate buffer, 137 mM Sodium Chloride and 2.7 mM Potassium Chloride) was purchased from Amresco; and Low-melting Agarose (LMA, Grade-Biotech) was purchased from Neta Scientific.

Detailed synthesis of hydrogel nanoparticles is disclosed in Jain, P., et al., “A Minimally Disruptive Method for Measuring Water Potential in Planta using Hydrogel Nanoreporters,” Proc Natl Acad Sci USA, 118(23) (2021) and U.S. Pat. No. 11,536,660 to Stroock, A., et al., the disclosures of which are incorporated herein by reference in their entirety. Briefly, the polyacrylamide (PAAm) nanoparticles were synthesized using an inverse microemulsion method. The aqueous polymerization solution contained AAm, BisAAm and APMA. Hexane, AOT and Brij30 were added and followed by sonication to form the microemulsion. The polymerization was triggered by adding APS and TEMED, and the dry nanoparticles were acquired by further removal of hexane, washing, precipitation and drying processes. Dry nanoparticles were resuspended into Sodium bicarbonate/Sodium Carbonate buffer. To the suspension, OG-NHS and RH-NHS dyes were dissolved in anhydrous DMF. The NHS-ester functional group on the dyes reacted with amine group on the nanoparticles for dye conjugation. The conjugated nanoparticles were again acquired by washing, precipitation and drying processes. Dry conjugated nanoparticles were resuspended in water through 20 minutes ultrasonication and were purified through centrifugation through concentrators (Pierce™ Protein Concentrators PES, 100K MWCO, 0.5-10 mL, Thermo Fisher Scientific) for eight rounds (5000 RCF, 15° C., 25 minutes).

The fabrication of the optical sensor patch using the hydrogel nanoparticles is the same as with the dye-based method, as described above, except for changing the 27.5 μl dye solution into 25 μl 100% (40 mg hydrogel nanoparticles/8 ml DI water) hydrogel nanoparticle solution depicted in FIG. 8.

### Example 3—Characterization Methods

Changes in the emission spectra from the optical sensor patch are characterized as a function of water activity with which the sheet is in equilibrium. A relative intensity index is extracted as a function of activity, ζ(a_{w}). This index is based on spectral decomposition of the total, background-subtracted emission spectrum of the optical sensor patch into the contributions from each of the dyes. The calibration curve is used, along with spectrally resolved measurements of the fluorescence from the optical sensor patch, to infer the water activity of the material's local environment.

Hydrogel nanoparticles have been used for two types of measurements, as described herein: (1) temporally resolved, spatially localized measurements of a_{w} in which a volume of the optical sensor patch is coupled to an optical fiber probe and submerged or embedded in a phase of interest; and (2) temporally and spatially resolved measurements of a_{w} in a material of interest over with which the optical sensor patch has been placed in contact.

### Example 4—Measurement of Water Activity

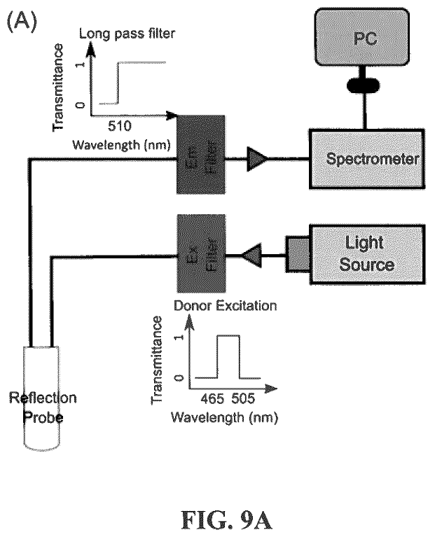

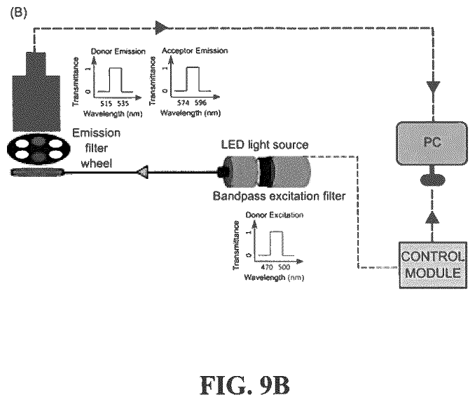

Instrumentation for using the optical sensor patch of the present technology is illustrated, for example, in FIG. 9A for performing point probe measurements and FIG. 9B using an imaging method. Both methods utilized the vacuum chamber described below.

### Vacuum Chamber

For the measurements on vapor water potential, a cured optical sensor patch of the present technology was placed in a temperature-controlled vacuum chamber with a control on vapor water activities, and the optical sensor patch was able to reach the equilibrium with the water activity in the vacuum chamber. The vacuum chamber system has been described, for example, in Vincent, O., et al., “Imbibition Triggered by Capillary Condensation in Nanopores.” Langmuir, 33(7): 1655-1661 (2017), and Vincent, O., et al., “Drying by cavitation and poroelastic relaxations in porous media with macroscopic pores connected by nanoscale throats.” Phys Rev Lett, 113(13): 134501 (2014), the disclosures of which are incorporated by reference herein in their entirety. The sample of the optical sensor patch was placed on a layer of white filter paper (No. 1, Whatman) to enhance the signal intensities.

### Point Probe for Temporally Resolved, Spatially Localized Measurements

Referring to FIG. 9A, a mercury light source (EL6000, Leica) was used as light source, and the light passed through a band-pass filter (465˜505 nm, Chroma Technology Corporation) to select the excitation light wavelength. The excitation light was emitted through six fiber bundles, and the reflection emission light was collected through the central single light fiber in the same bundle (QR600-7-UV-125F, Premium 600-micron Reflection Probe, Ocean Optics Inc.). The reflection emission light passed through a high-pass filter (>510 nm, Chroma Technology Corporation) to avoid bleeding from the excitation light. The acquired light was sent to the spectrometer (Ocean Optics Inc., ST2000) and saved by Ocean View software operating with an integration time of 1˜4 seconds to have proper signal intensity.

The relative intensity of fluorescence was measured using spectra deconvolution. The acquired spectra were composed of the relative contribution of the emissions of both dyes (OG and Rho), which can be represented as Equation (1), as described in Jain, P., et al., “A Minimally Disruptive Method for Measuring Water Potential in Planta using Hydrogel Nanoreporters,” Proc Natl Acad Sci USA, 118(23) (2021) and U.S. Pat. No. 11,536,660 to Stroock, A., et al., the disclosures of which are incorporated herein by reference in their entirety:

$${Em}_{e xp} = {xEm}_{OG} + {yEm}_{Rho} \qquad ( 1 )$$

where, Emₑₓₚ is the emission spectrum acquired from each measurement, Em_{OG} and Em_{Rho} are the normalized emission spectra provided by the chemical vendor; x is the emission contributed by OG dye and y is the emission contributed by Rho dye. The contribution x and y can be determined through deconvolution through ‘lsqnonneg’ function in the MATLAB software. Thus, the relative intensity index (ζₑₓₚ) can be calculated through Equation (2):

$${ζ}_{e xp} = \frac{y}{x + y} \qquad ( 2 )$$

### Imaging System Measurements

An exemplary imaging system for performing temporally-resolved, spatially localized measurements using the optical sensor patch is shown in FIG. 9B. The measurements were performed in the same temperature-controlled vacuum chamber described previously.

The imaging system included: (1) an in-house built LED light source, (2) a CCD camera with lens, and (3) the Arduino-driven automatic filter wheel. A 10 W cyan LED (central wavelength=490 nm, CHAZHAN) was connected to a constant current transistor (constant current=900 mA, CHAZHAN) for AC to DC conversion. The emitted light passed through a band-pass filter (470˜500 nm, Chroma Technology Corporation) to select the excitation wavelength, and then the excitation light was transmitted through a ring light (MA25, Dolan-Jenner) to illuminate sample evenly. The emission images were acquired by an FLIR CCD camera (BFS-U3-04S2C-C, Teledyne FLIR) attached with a lens (f/2.8˜56, 60 mm focal length, Nikon). An Arduino-driven automatic filter wheel (LCFW5, ThorLabs) was placed between the sample and CCD camera, with two band-pass filters (515˜535 nm for OG emission channel or 574˜596 nm for Rho emission channel, respectively, Chroma Technology Corporation) shifted automatically for acquirement at OG or Rho emission channel.

The relative intensity of the fluorescence spectra was measured as disclosed in Gadella, T.W.J., “FRET and FLIM Techniques.” LABORATORY TECHNIQUES IN BIOCHEMISTRY AND MOLECULAR BIOLOGY, ed. S. Pillai; and P.C.v. Vilet. Vol. 33. ELSEVIER (2009), the disclosure of which is incorporated herein by reference in its entirety.

To determine the relative intensity, a set of two images were acquired (exposure time=1˜4 seconds) for analysis. The first image

$$( {F}_{O {G}_{e x}}^{O {G}_{e m}} )$$

was captured with OG excitation channel and detected at OG emission channel; the second image

$$( {F}_{O {G}_{e x}}^{R h {o}_{e m}} )$$

was captured by being excited at OG excitation channel and collected at Rho emission channel. In each pixel i, the intensities,

$${{F}_{{D}_{e x}}^{{D}_{em}}}_{i} and {{F}_{{A}_{e x}}^{{D}_{em}}}_{i} ,$$

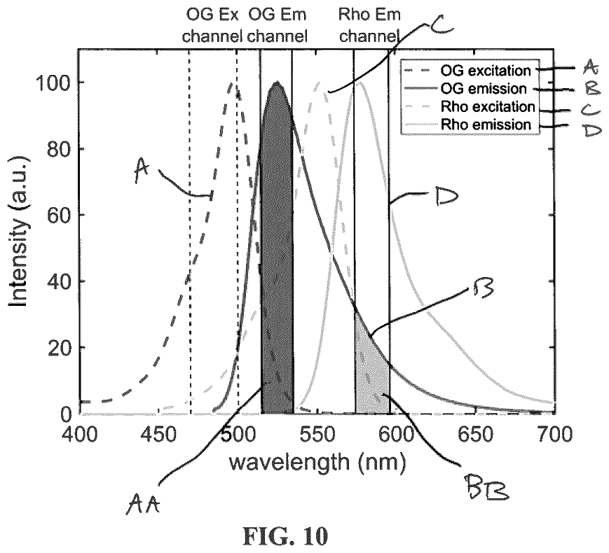

were recorded as grayscale value for pixelwise intensity evaluation. However, in this imaging method, bleed-through is considered as the OG emission contribution by OG excitation collected at the Rho emission window. This bleed-through contribution (shaded area AA under OG emission curve depicted in FIG. 10) is not considered as the Rho emission. Thus, a bleed-through correction factor, a_{c} in Equation (3), which is the ratio of integrated area in Em_{OG} at Rho emission channel (shaded area AA under OG emission curve depicted in FIG. 10) to integrated area in Em_{OG} at OG emission channel (shaded area BB under OG emission curve depicted in FIG. 10) is introduced for subtraction to correct the relative intensity.

$${α}_{c} = \frac{{Area}_{o {G}_{e x}}^{R h {o}_{e m}}}{{Area}_{O {G}_{e x}}^{O {G}_{e m}}} \qquad ( 3 )$$

As the result, the pixelwise relative intensity (ζ_{corr,i}) can be calculated as Equation (4):

$${ζ}_{corr , i} = \frac{{{F}_{{OG}_{e x}}^{{Rho}_{em}}}_{i} - {α}_{c} \times {{F}_{O {G}_{e x}}^{O {G}_{em}}}_{i}}{{{F}_{O {G}_{e x}}^{O {G}_{em}}}_{i} + {{F}_{O {G}_{e x}}^{R h {o}_{e m}}}_{i} - {α}_{c} \times {{F}_{{OG}_{e x}}^{O {G}_{em}}}_{i}} \qquad ( 4 )$$

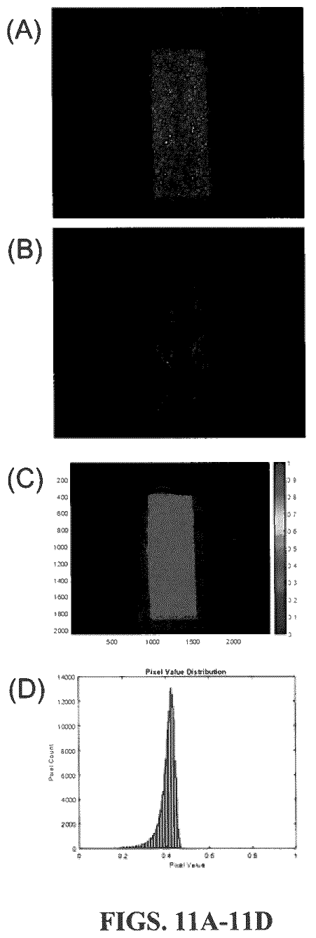

The analysis of the spatial distribution of relative intensity is illustrated in FIGS. 11A-11D. As discussed above, a set of two images were acquired

$${F}_{O {G}_{e x}}^{O {G}_{e m}}$$

### (FIG. 11A) and

$${F}_{{A}_{e x}}^{{D}_{em}}$$

(FIG. 11B) for analysis. The pixelwise relative intensity (ζ_{corr,i}) can be calculated through Equation (4), and FIG. 11C showS the spatial distribution of ζ_{corr,i} for the corresponding

$${{F}_{O {G}_{e x}}^{O {G}_{em}}}_{i} and {{F}_{{OG}_{e x}}^{R h {o}_{em}}}_{i} .$$

For one measurement of steady-state characterization, a pixel count distribution of calculated ζ_{corr},i will be plotted (as shown in FIG. 11D), and the median of this distribution will be determined as the ζ_{corr} corresponding to this measurement steady-state measurement.

### Example 5—Results (Hydrogel Nanoparticle Based Optical Sensor Patch)

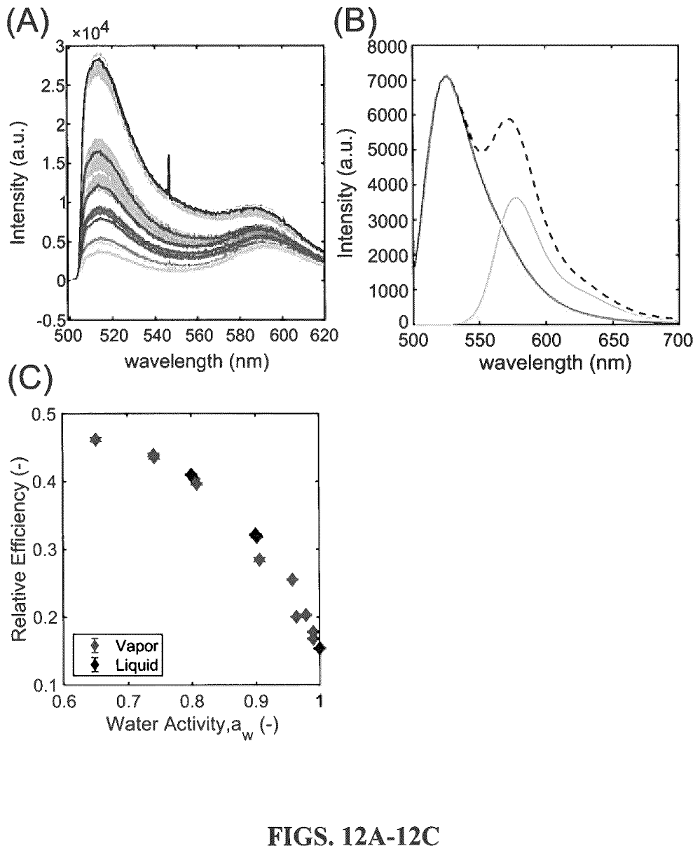

Characterization of the hydrogel nanoparticle-based optical sensor patch was performed through both vapor and liquid water activity, as illustrated in FIGS. 12A-12C. In FIG. 12A, a set of spectra was depicted to show the raw spectra change with the change of water activity. The blue spectra showed the emission spectra of the hydrogel nanoparticle-based optical sensor sheet in equilibrium close to saturation (a_{w}˜1). When the spectrum were with more yellowish color, it represented the emission with the decrease of the water activity away from the saturation (a_{w}<1).

FIG. 12B represents a cased of spectrum deconvolution with the method discussed above. This deconvolution can be beneficial for understanding the relative contribution of OG and Rho dyes for a collected emission spectrum. The vapor water activity characterization was performed in the temperature-controlled vacuum chamber system discussed above. The liquid water activity characterization was performed through submerging the optical sensor patch into the solution. In FIG. 12C, the characterizations for both liquid and vapor water activity provided a robust response across a range of water activity.

### Transient of the Optical Sensor Patch

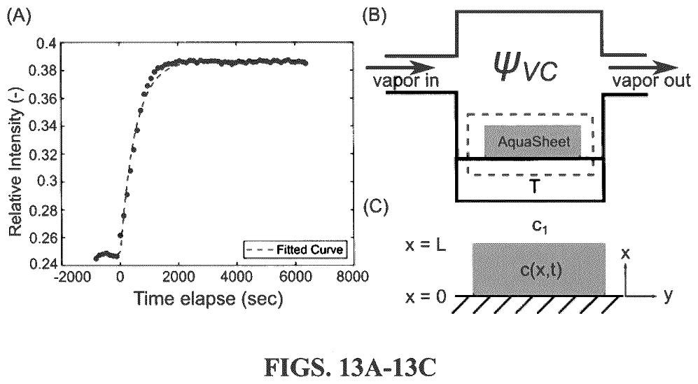

The transient of a sensor represents the relaxation time needed to reach the equilibrium with the environment. This transient is an important sensor characteristic because it represents the time required for each measurement and the ability to capture the temporal change of water activity. To capture the transient of the optical sensor patch, a step change of water activity in the vacuum chamber was applied and sets of images were acquired in fixed time interval (Δt=2 minutes) to present the evolution of relative efficiency as shown in FIG. 13A. The time constant of the transient was extracted based on an exponential fit as shown in Equation (5):

$$I ( t ) = Δ {I}_{0} \times {e}^{- \frac{t}{{τ}_{A q S}}} \qquad ( 5 )$$

Where t represents the elapse time, I(t) is the relative intensity at time t, ΔI₀ represents the amplitude and τ_{AqS} is the time constant. According to this fitting, the time constant (τ_{AqS}) equals to 537 seconds as through the fitting in FIG. 13A (dashed line), and this value reveals the characteristic of transient of the optical sensor patch.

To understand the nature of this transient behavior, a 1D diffusion-limit model was used to describe the transient of the optical sensor patch. The 1D diffusion equation in Equation (6) can describe this transient behavior:

$$\frac{\partial c}{\partial t} = D \frac{{\partial }^{2} c}{\partial {x}^{2}} \qquad ( 6 )$$

where c is the concentration of vapor or liquid water, and D is the diffusivity of water vapor in the optical sensor patch matrix. It is assumed that the optical sensor patch already reached the previous equilibrium, and thus the initial concentration in the optical sensor patch is c₀. According to the schematic diagram of the optical sensor patch sample in the vacuum chamber in FIGS. 13B and 13C, no mass flux condition,

$$\frac{\partial c}{\partial x} ( 0 , t ) = 0 ,$$

is applied at the bottom, and a constant vapor concentration, c(x=L)=c₁, is imposed at the top surface which exposed to the vapor which defined the water activity in the vacuum chamber. The analytical solution for Equation (6) with these boundary conditions will be Equation (7):

$$\frac{c ( x , t ) - {c}_{1}}{{c}_{0} - {c}_{1}} = {\sum }_{n = 0}^{∞} \frac{2}{( n + \frac{1}{2} ) π} sin [ ( n + \frac{1}{2} ) π ] {e}^{- \frac{{( n + \frac{1}{2} )}^{2} {π}^{2} D}{{L}^{2}} t} cos [ \frac{( n + \frac{1}{2} ) π x}{L} ] \qquad ( 7 )$$

The first term (n=0) will dominate the transient. Thus, the time constant will be calculated as Equation (8):

$${T}_{A q S} = \frac{{L}^{2}}{{( n + \frac{1}{2} )}^{2} {π}^{2} D} \qquad ( 8 )$$

The thickness L of the optical sensor patch is 0.8 mm, and the diffusivity D as 10⁻⁹ m²/s is used as disclosed in Watson, J.M., et al., “A study of organic compound pervaporation through silicone rubber.” Journal of Membrane Science, 49(2): p. 171-205 (1990), the disclosure of which is incorporated by reference herein in its entirety. When n=0, the theoretical time constant of the optical sensor patch is equal to 260 seconds.

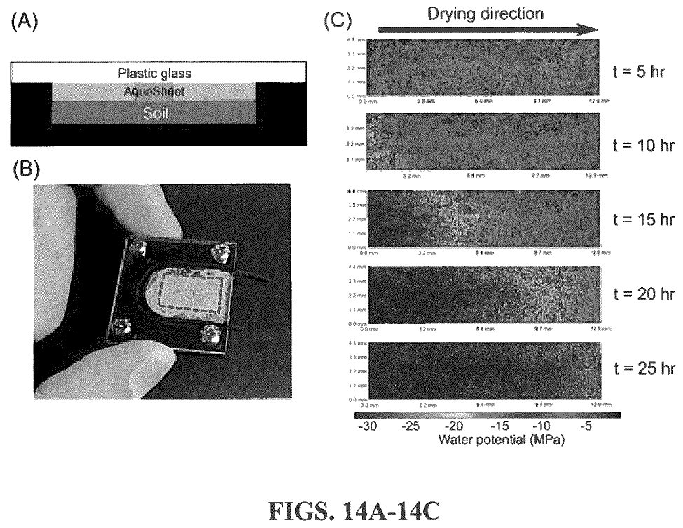

The hydrogel nanoparticle-based optical sensor patch can be used to report temporally and spatially resolved water activity. The optical sensor patch can be used to capture the spatiotemporal distribution of water activity in capillary-driven processes in porous media. Among various porous media, soil is considered one of the most challenging because of chemical and structural heterogeneity. FIGS. 14A-14C illustrate a drying experiment to investigate the evolution of water status in soil by the optical sensor patch. An optical cell was designed to observe the drying process with the optical sensor patch as shown in FIGS. 14A and 14B. The spatiotemporal evolution is shown in FIG. 14C. This result revealed the ability of the optical sensor patch to capture the water status distribution in porous media, enabling direct measurements on water status (in contrast to conventional tools to measure water content) for the first time.

### Example 6—Results (Free Dye Based Optical Sensor Patch)

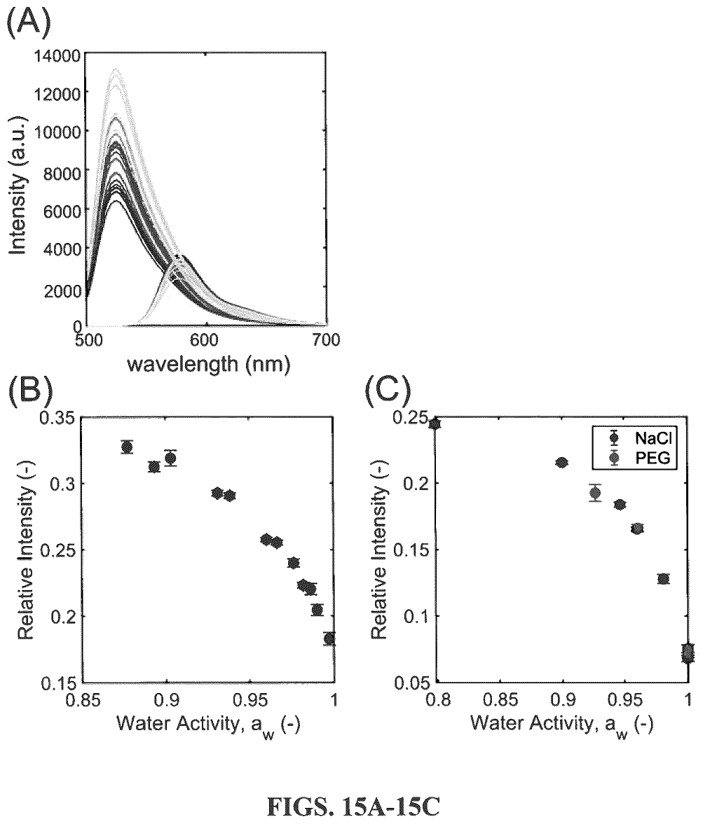

The response of the optical sensor patch employing free dyes to vapor water activity in the temperature-controlled vacuum chamber system is characterized, as shown in FIGS. 15A and 15B. In FIG. 15B, the decomposed OG (emission peak: 520 nm) and Rho (emission peak: 568 nm) emission spectra are plotted based on the deconvolution results. The spectra were collected with decreasing water activities from yellow to blue. It is inferred that OG showed quenching in response to the decreasing water activity; in contrast, Rho provided relatively constant emission in response to decreasing water activity. A robust response was observed over the range from −0.5 to −20 MPa.

The response of the optical sensor patch to changes in water potential in aqueous solutions is further characterized. Importantly, consistent response to solutions of different solutes was observed as shown in FIG. 15C. The optical sensor patch captured the water potential in two different solutions (sodium chloride (NaCl) and polyethylene glycol (PEG)) with varying water potential in the same manner.

### Example 7—In Vitro Measurements

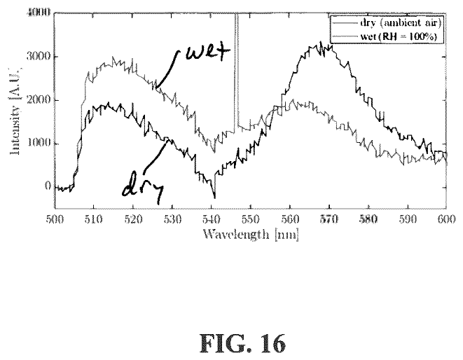

As an initial test, on optical implant was subjected to two different conditions: (1) exposure to ambient air and (2) immersion in a water solution. Due to the distinct water potential in these two conditions, the fluorescence spectrum captured by the optical fiber differs as expected from the behavior of the hydrogel nanoparticles, as shown in FIG. 16. For this experiment, an implant with a double-patch design was used. The first window contained an optical sensor patch, while the second window contained pure PDMS, serving as a background signal.

### Example 8—In-Planta measurements

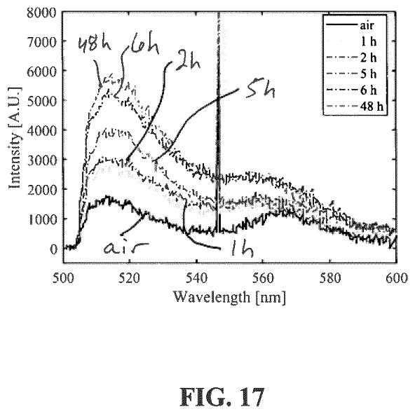

A dry-down experiment was conducted on a tomato plotted in surface (allowing fast dry down), using the same batch of implants that were used for the in-vitro measurements. FIG. 17 shows a time lapse of the spectral response of the implant during the dry-down experiment.

### Example 9

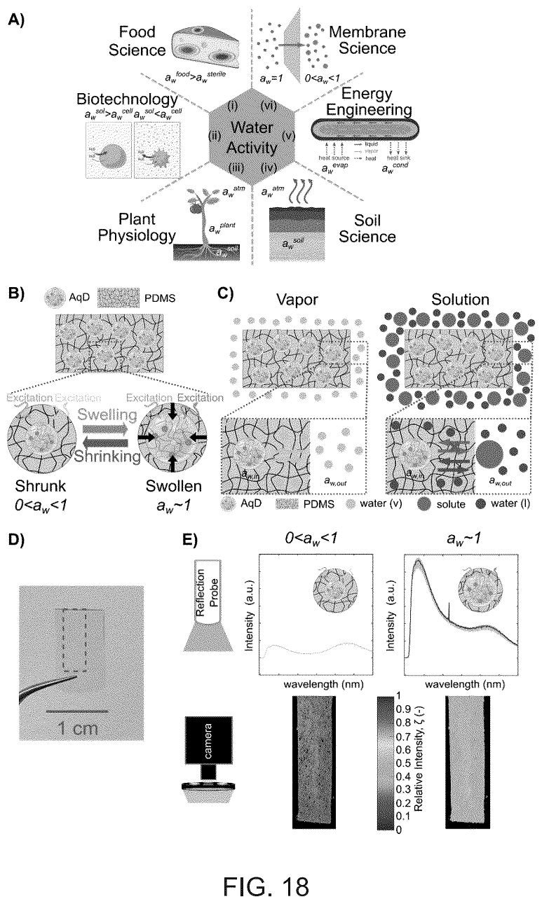

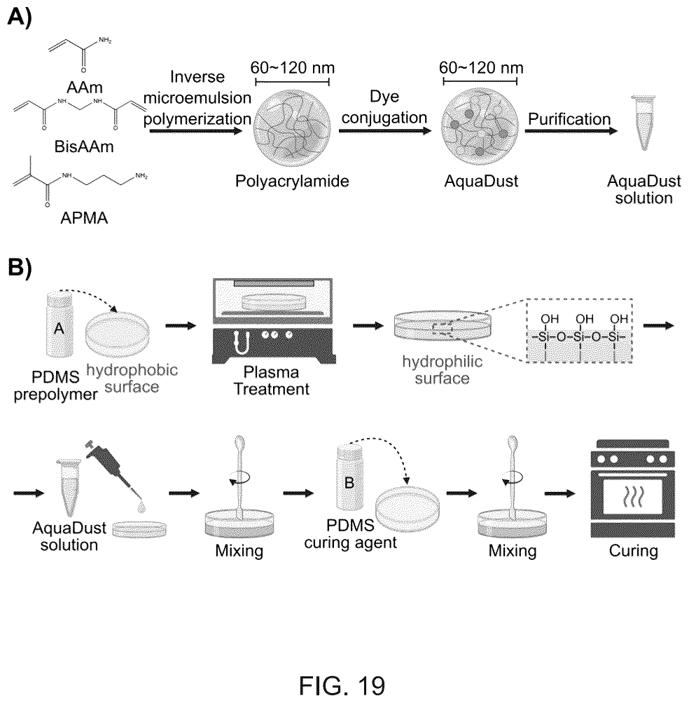

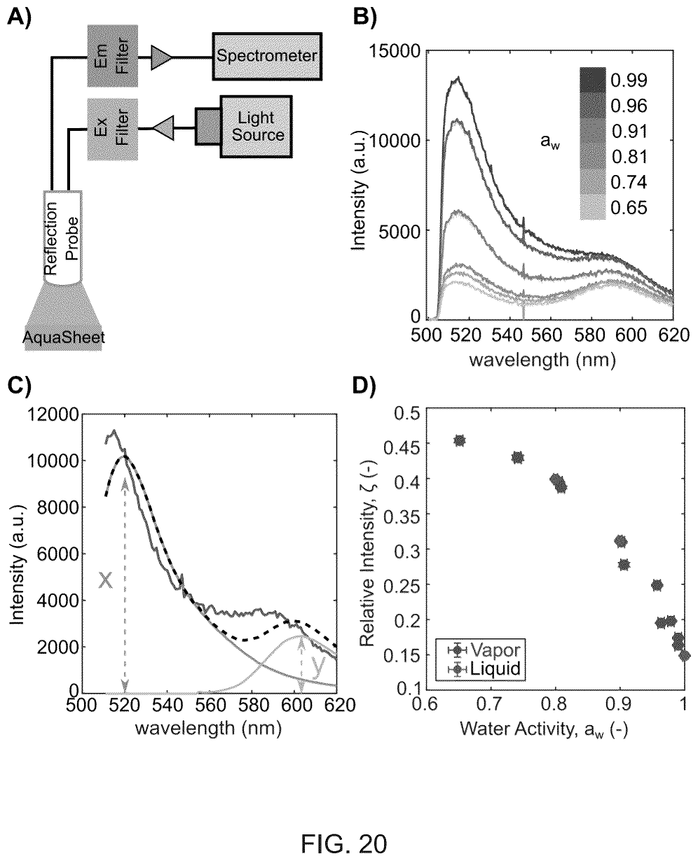

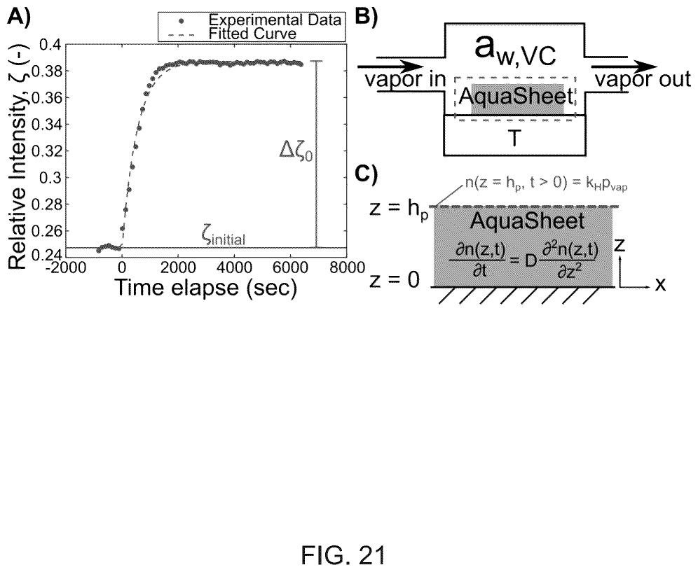

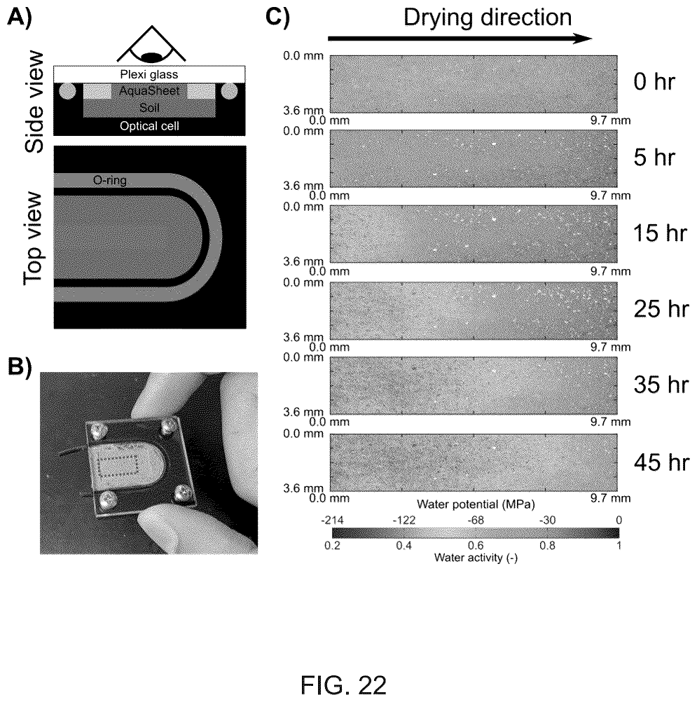

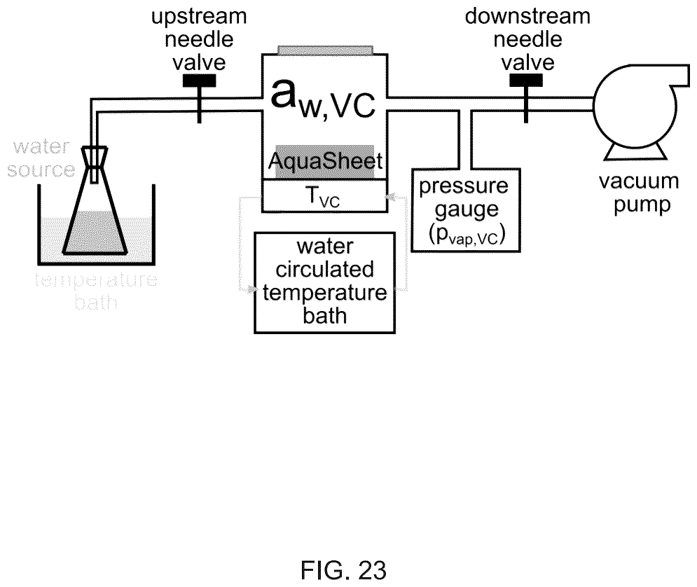

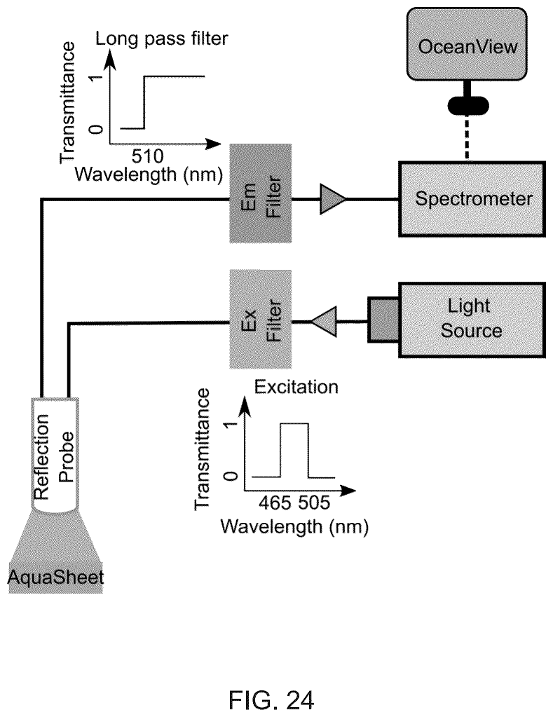

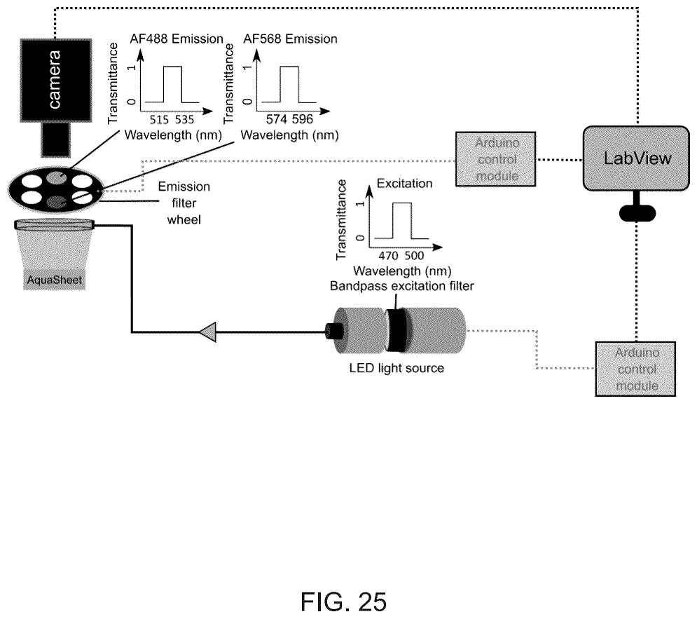

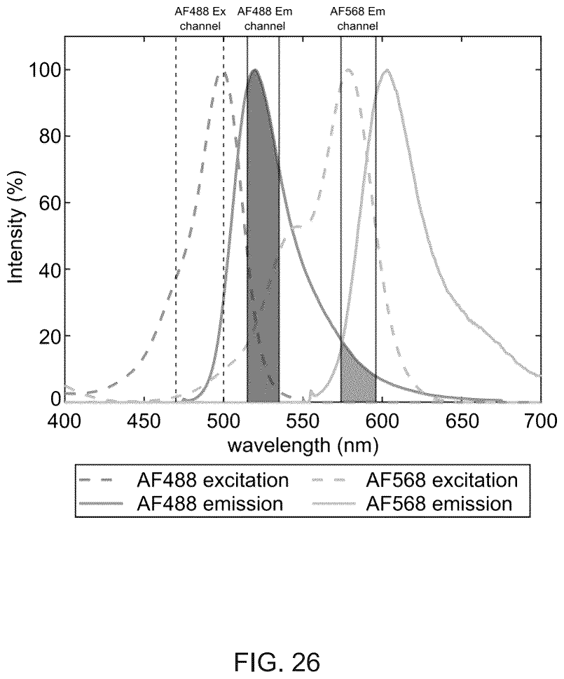

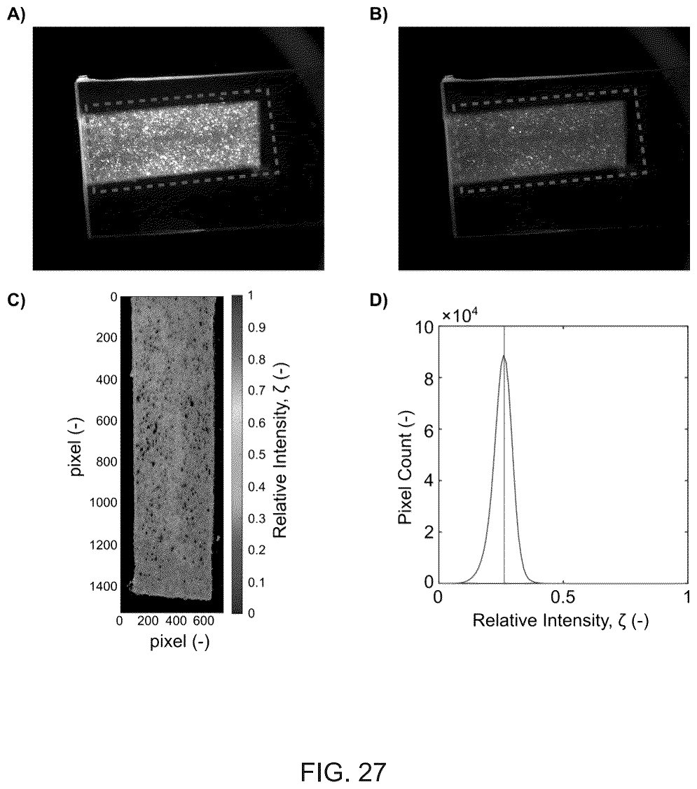

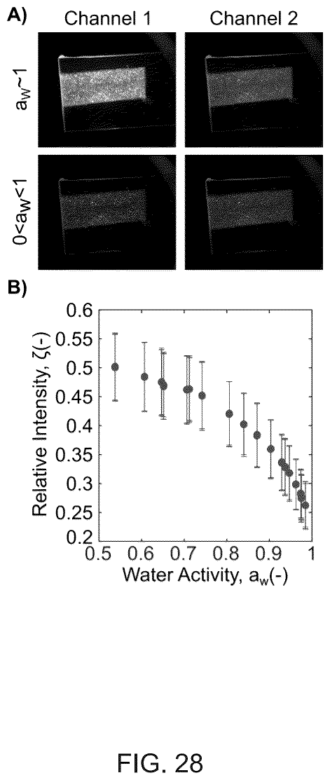

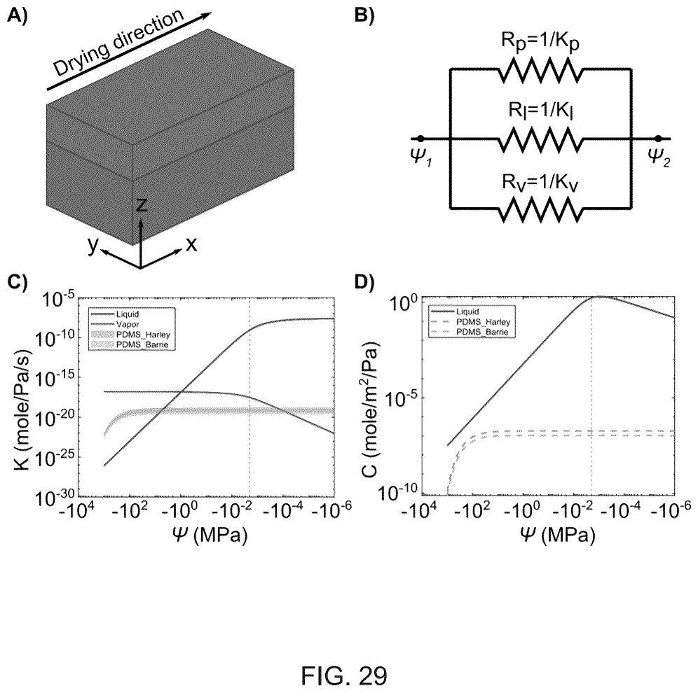

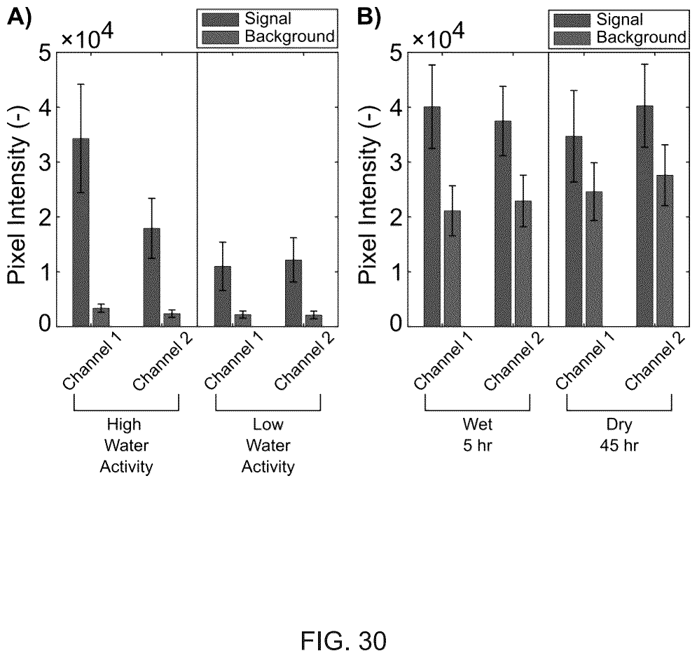

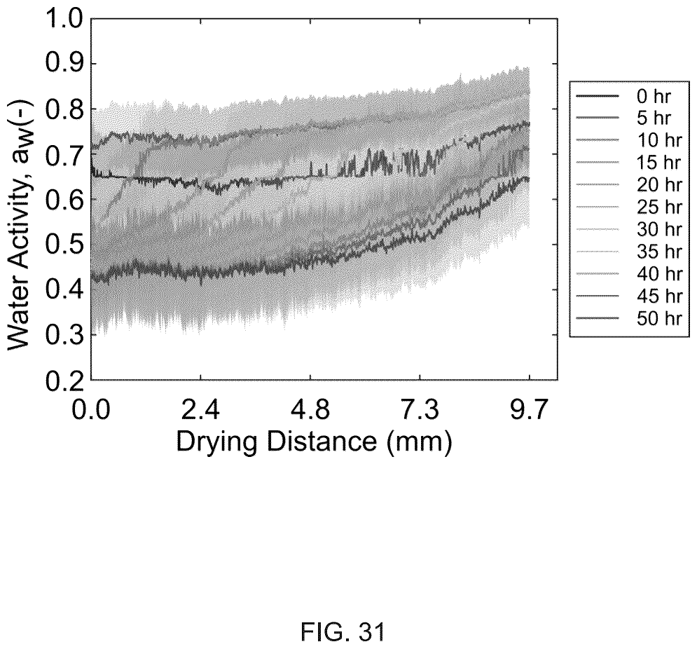

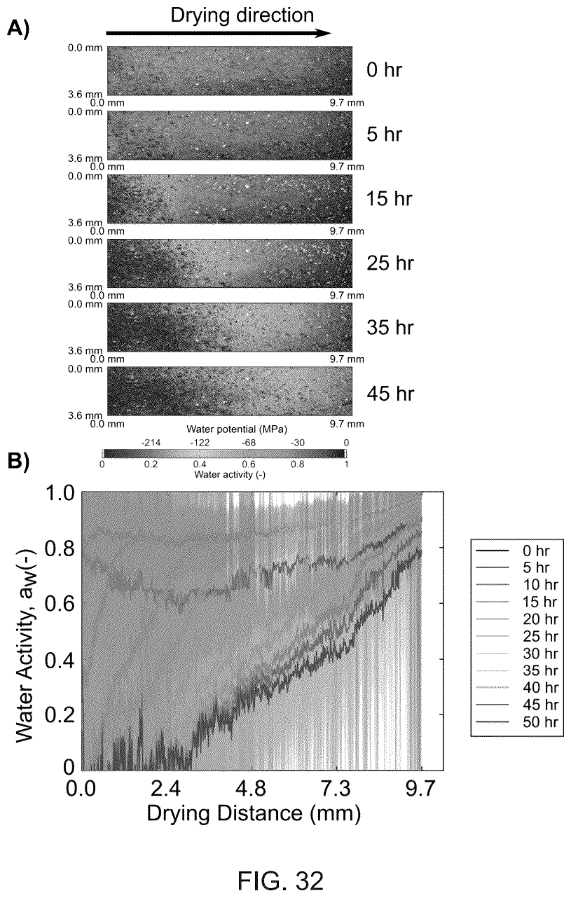

Water plays important roles in physical, chemical, and biological processes in nature and technology. Water activity (equivalently, water potential, relative humidity, fugacity, or the chemical potential of water) represents the thermodynamic availability of water to participate in these processes, as depicted in FIG. 18(A). Water activity defines sterility and controls the preservation of the flavor and texture of food products. Water activity is highly regulated in mammalian tissues and must be delicately controlled in biological processes such as cell culture. Water activity in the environment (relative humidity in the atmosphere and water potential in the soil) controls the hydraulic status of plants and thus plant health, productivity, and efficiency of water-use. The activity of water below ground influences geological processes, geotechnical systems, and agricultural management. In other technological contexts, such as energy management systems (e.g., heat pipes as shown), separation membranes, and synthetic porous media, water activity controls phase-change, fluid-material interactions, and transport processes. Despite its central importance in this diversity of contexts, there is still a lack of robust and versatile tools to measure water activity in various states and environments.

A number of technologies exist to measure water activity. For ex situ, benchtop measurements, chilled mirror hygrometers can provide high accuracy, rapid measurements of the water activity of complex samples, and freezing point osmometers serve for liquid samples near saturation (a_w˜1). Numerous designs of hygrometers for point measurements of vapor activity sense, for example, changes in the electrical resistance or capacitance of a working material; these “relative humidity” sensors have modest accuracy (δa_w=±0.02) and are not directly compatible with in situ applications in condensed phases. Thermal couple psychrometers have been used for both in situ and ex situ point measurements in a wide range of contexts. However, these measurements involve a complicated transient signal and high sensitivity to temperature gradients. Conventional tensiometers provide exceptional sensitivity for point measurement near saturation and have been particularly useful for in situ measurements in soil. A microelectromechanical systems (MEMS) design tensiometer developed in our previous publications can serve for both in and ex situ measurements of water activity and has found application for in situ measurements in plant tissues.

Despite these developments on measuring water activities, there are still outstanding challenges in various applications. These challenges include ensuring environmental compatibility for in situ point measurements in both vapor and chemically complex condensed phases, where packaging sensors with electronic components is particularly difficult. Additionally, these methods struggle with versatility in integration into complex geometries and with diverse materials, which is essential for applications such as plant physiology and soil science. Furthermore, there is a need for these techniques to provide spatial and temporal measurements of water activity, which is crucial for monitoring the evolution of processes in environmental contexts, such as soils, and in manufacturing contexts, such as the curing of materials or the aging of foods.

This disclosure reports a composite, AquaSheet, to perform: (i) temporally resolved, spatially averaged measurements and (ii) temporally and spatially resolved measurements of water activity in complex environments.

Material design: FIG. 18(B-C) shows the design of AquaSheet.

FIG. 18(B) depicts the dispersion of nano-sized hydrogel particles, AquaDust, in a thin sheet of silicone elastomer (PDMS) to form a material with a fluorescence spectrum that depends on the activity of water, a_w, with which it is at equilibrium. The composite material is referred to as “AquaSheet”. Each hydrogel nanoparticle contains pairs of fluorescent dyes covalently linked to the polymer matrix. “AquaDust” particles and their fluorescence response to changes in water activity have been previously described. The spectral changes of AquaSheet fluorescence is interpreted as a function of water activity as due to a combination of changes in self-quenching and changes in Förster Resonance Energy Transfer (FRET) between the pairs of dyes with changes in the hydration state of the system. Importantly, the inclusion of two dyes allows for the definition of a ratiometric response that mitigates well-known artifacts in fluorescence measurements.

The encapsulation of the AquaDust particles in AquaSheet provides a water-selective barrier that protects the particles from the chemical environment in which it is embedded while allowing for transfer and equilibration of water. This encapsulation strategy follows that reported for environmental sensing of oxygen based on quenching of phosphorescence dyes; these reporter systems have sometimes been called, “Optodes” in the literature. In vapor, FIG. 18(C) depicts the transfer of water molecules through the silicone matrix to reach an equilibrium of water activity both in (a_(w,in)) and out (a_(w,out)) the matrix, allowing the measurements of water activity in vapor phase. In solutions, FIG. 18(C) also depicts the selectivity of the silicone matrix, with the transfer of water molecules and the exclusion of solutes that are insoluble in the matrix (e.g., ions and polar molecules). This selectivity should allow for the measurement of water activity in aqueous solutions of polar solutes. The encapsulation also isolates the reporter particles from interactions with solid substrates that can influence their response to water activity.

Measurements and Responses: FIG. 18(D-E) shows the measurements and responses of AquaSheet

In FIG. 18(D), a piece of AquaSheet sample is shown. This silicone-based composite provides mechanically flexibility and robustness for repeated use in a variety of contexts.

The use of AquaSheet was explored with two methods of fluorescent measurement. The top row of FIG. 18(E) presents a reflectance probe system for the acquisition of fluorescence emission spectra for temporally resolved, spatially averaged measurements. The swollen (a_w˜1) and shrunk (0<a_w<1) states of AquaDust in AquaSheet provide fluorescence emission spectra, respectively, which is quantified with a relative intensity index (ζ) based on the relative contribution of two dyes. The lower row of FIG. 18(E) presents the imaging method for temporally and spatially resolved measurements. The relative intensity index (ζ) can be calculated by the ratiometric calculations considering the fluorescence intensity at the emission peaks of two dyes in response to the water activities.

This disclosure presents the design and fabrication of AquaSheet, a novel technology for measuring water activity. It is characterized and calibrated for its response in both vapor and liquid solutions with a commercial reflectance probe spectrometer and a custom fluorescence ratiometric imaging system. Additionally, the use of AquaSheet is demonstrated to track the spatial and temporal evolution of water activity in a sample of soil undergoing drying.

### Methods and Materials

### Materials

Acrylamide (AAm, 40% (w/v)), N,N-methylene bisacrylamide (BisAAm, >98%),

Ammonium Persulfate (APS, >99.99%), Tetramethylethylenediamine (TEMED, Electrophoresis grade) were purchased from Fisher Scientific; N-aminopropyl methacrylamide (APMA, >98%) was purchased from Polysciences Inc; Dioctyl Sulfoccinate Sodium salt (AOT, 96%) and Polyoxyethylene(4)lauryl ether (Brij30) were purchased from ACROS Organics; n-Hexane (95%, HPLC Grade) was purchased from Millipore Sigma; N,N-Dimethyl formamide (DMF, Anhydrous) was purchased from Mallinckrodt Inc.; Alexa Fluor 488 N-hydroxysuccinimidyl ester (AF488-NHS) and Alexa Fluor 568 N-hydroxysuccinimidyl ester (AF568-NHS), were purchased from Thermo Fisher Scientific; Ethanol (Anhydrous, 100%) and Isopropyl alcohol (IPA) (99%) were purchased from VWR International; Phosphate-buffered saline (PBS) 1× tablet (10 mM Phosphate buffer, 137 mM Sodium Chloride and 2.7 mM Potassium Chloride) were purchased from Amresco.39,40 Polydimethylsiloxane (PDMS) elastomer kit (Sylgard 184) was purchased from Dow Corning.

### Method

The synthesis of AquaDust:

The detailed synthesis method was presented in U.S. Pat. No. 11,536,660, hereby incorporated by reference in its entirety. FIG. 19(A) shows a brief schematic diagram of the synthesis: the polyacrylamide (PAAm) nanoparticles were synthesized using an inverse microemulsion method. The aqueous polymerization solution (2 ml) was formed with: 300 μl AAm solution, 144 μl BisAAm solution (3.6 mg/144 μl H2O) and 240 μl of APMA solution (12 mg/240 μl H2O). Hexane (42 ml), AOT (1.2 g) and Brij30 (2.88 ml) were added and followed by sonication (FS60H, Fisher Scientific; 3 minutes, 40 kHz) to form the microemulsion. The polymerization was triggered by adding 50 μl of APS solution (10 mg/50 μl H2O) and TEMED (25 μl) to the microemulsion. Purified nanoparticles were recovered by removal of hexane by rotary evaporation, five rounds of washing by ethanol (15˜25 ml), precipitation through centrifugation (8000 RCF, 15° C., 5 minutes), and drying under vacuum for 25 minutes. Dry nanoparticles were resuspended into Sodium bicarbonate/Sodium Carbonate buffer (pH 8.3). Conjugation of the dyes was performed as follows: 5 mg AF488-NHS dye was dissolved in 500 ml anhydrous DMF and 15 mg AF568-NHS dye was dissolved in 1500 ml DMF. Two solutions were mixed and added to the nanoparticle suspension. The NHS-ester functional group on the dyes reacted with amine group on the nanoparticles for covalent conjugation. The conjugated nanoparticles were again recovered through five rounds of washing by ethanol (15˜25 ml), precipitation through centrifugation (8000 RCF, 15° C., 5 minutes) and drying by vacuum for 25 minutes. Dry conjugated nanoparticles were resuspended in water through 20 minutes ultrasonication and were purified through centrifugation through concentrators (Pierce™ Protein Concentrators PES, 100K MWCO, 0.5˜10 mL, Thermo Fisher Scientific) for eight rounds (5000 RCF, 15° C., 25 minutes).

AquaSheet fabrication:

FIG. 19(B) illustrates the principal steps in the fabrication of AquaSheet: 0.25 g of PDMS precursor was measured in a petri-dish (35 mm in diameter, Corning) and allowed 5 minutes to spread by gravity into a layer of ˜2 cm in diameter and ˜0.2 cm in thickness. Then, the surface of PDMS precursor was oxidized in a low temperature air plasma (PDC-001, Plasma Cleaner, Harrick) at 200 Watts for 4 minutes to increase the hydrophilicity of the surface.54.55 Immediately after removing the precursor from the plasma chamber, 25 μl 100% (40 mg AquaDust/8 ml deionized water) AquaDust solution was pipetted onto the surface of the oxidized PDMS precursor. Mixing was performed manually with spatulas until the mixture became uniformly cloudy and no obvious dye solution floated on the surface (˜5 minutes). Next, 0.025 g of PDMS curing agent was added into the AquaDust/precursor mixture (at a 10:1 precursor to curing agent ratio) and mixed manually again for 5 minutes. The mixture was then degassed in a vacuum desiccator with a vacuum pump (RV12, Edwards) for 20 minutes.

To define the thickness of cured AquaSheet, a pristine PDMS (with same precursor to curing ratio as 10:1) with the same thickness was fabricated, and a circular mold was cut with the AcuPonch Biopsy Punch (5 mm in diameter, Acuderm Inc.). The PDMS mold was placed onto a new petri-dish (100 mm in diameter, Falcon), and the degassed uncured AquaSheet was cast into the well until the meniscus reached the same level as PDMS mold. Uncured AquaSheet was cured under 50° C. for two days avoiding the formation of bubbles due to the rapid evaporation of water and followed by 80° C. for one day to cure AquaSheet thoroughly.

AquaSheet response to changes in water activity

Mechanism of AquaSheet response: FIG. 18(B) depicts the mechanism by which AquaSheet responds to changes in the water activity in its local environment. The silicone matrix serves as a selective barrier around the AquaDust, allowing water molecules to permeate the matrix and interact with AquaDust nanogels. AquaDust responds to changes in water activity by swelling and shrinking of the hydrogel matrix. When AquaDust equilibrates with a higher water activity and swells, the effective concentration of the embedded dyes decreases; when it equilibrates with a lower water activity and shrinks, the effective concentration of dyes increases. These changes in the concentration of the two dyes affects their fluorescence emission spectra in at least two ways: First, lower concentrations (higher water activity, swelled state) increase the intermolecular distance between the dyes, leading to a decreased FRET efficiency; conversely, higher concentrations (lower water activity, shrunk state) decrease the intermolecular distance between the dyes, leading to an increased FRET efficiency. Second, as is well established in the literature, many dyes display concentration-dependent quenching, such that their emission intensities decrease at higher concentration. The date in FIG. 20(B) suggests that the response of silicone-encapsulated AquaDust is likely a combination of FRET behavior and self-quenching of the dyes processes, or a combination thereof

Measurements and characterization of water activities

Vacuum chamber for water activities measurements:

For measurements in vapor, a cured AquaSheet sample was placed on the temperature-controlled floor of a vacuum chamber (FIG. 23). This vacuum chamber system has been described previously. Briefly, a vacuum pump drove the water vapor from a source reservoir into the chamber and steady values of the vapor pressure were set by adjusting two needle valves, one upstream and one downstream of the chamber. A vacuum gauge attached to the chamber measures the pressure, p_(vap,VC). The water activity in the chamber (a_(w,VC)) was assessed based on the saturation vapor pressure (p_vap\hat{ }sat (T)) at the measured temperature of the floor of the chamber with which the sample is in contact:

$${a}_{w , VC} = \frac{{p}_{v a p , V C}}{{p}_{v a p}^{sat} ( T )} \qquad ( 1 )$$

Instrumentation for point measurements is shown in FIG. 24

Reflectance probe for temporally resolved, spatially averaged measurements:

A mercury lamp (EL6000, Leica) was used as light source with a band-pass filter (465˜505 nm, Chroma Technology Corporation) to select the excitation light wavelengths. The excitation light was delivered through six-fiber bundle, and the emission light was collected through a single central light fiber in the same bundle (QR600-7-UV-125F, Premium 600-micron Reflection Probe, Ocean Optics Inc.). The sample of AquaSheet was placed on a layer of white filter paper (No. 1, Whatman) to increase the signal intensities because of the higher reflectance of white background. The emission light passed through a high-pass filter (>510 nm, Chroma Technology Corporation) to minimize bleed-through of the excitation light. The acquired light was sent to a spectrometer (Ocean Optics Inc., ST2000) and saved by Ocean View software operating with an integration time of 1˜4 seconds to achieve desired signal intensity: typically >5,000 counts to be much larger than noise and features in background spectrum and <60,000 counts to avoid saturation of the detector. Before the acquirement of the sample emission, a background mission was acquired through exciting the background filter paper under the same optical environment, and the background emission was subtracted through the “background subtraction” function in OceanView software.

Measurement and calibration of AquaSheet fluorescence response using reflectance probe.

Upon excitation with blue light, it is assumed that the acquired emission spectrum, Em_exp (λ) (dashed black curve in FIG. 20(C)), could be decomposed into contributions from the independent emission spectra of each dye (Em_AF488 (λ) and Em_AF568 (λ)—Egreen and yellow curves in FIG. 20(C)):

$$E {m}_{exp} ( λ ) = x E {m}_{AF 488} ( λ ) + y E {m}_{AF 568} ( λ ) . \qquad ( 2 )$$

In Eq. 2, Em_AF488 (λ) and Em_AF568 (λ) are the normalized emission spectra provided by the chemical vendor; x is the fractional emission contributed by AF488 and y is the fractional emission contributed by AF568. The relative contributions, x and y are determined by decomposition with the ‘lsqnonneg’ function in MATLAB. With these values, the relative intensity index (ζ_exp) is defined as:

$${ζ}_{exp} ( {a}_{w} ) = \frac{y}{x + y} \qquad ( 3 )$$

For each new batch of AquaSheet, a calibration was performed by assessing ζₑₓₚ(a_{w}) for samples in equilibrium with a series of known water activities in vapor or solution, spanning the range of interest (FIG. 20(D)).

Instrumentation of ratiometric imaging measurements is shown in FIG. 25. The measurements were performed on samples in the temperature-controlled vacuum chamber described previously (FIG. 23) or on the benchtop (soil drying experiment presented in FIG. 22).

Imaging system for temporally resolved, spatially resolved measurements: The imaging system included three parts: An LED light source, CCD camera with lens, and the Arduino-driven automatic filter wheel. A high-power light guide coupled LED source with a customized coupler serves as the light source (470_{±20 nm}, 50 W, Type-H, MIGHTEX). The excitation light passed through a band-pass filter (470˜500 nm, Chroma Technology Corporation) to select the excitation wavelength, and then the excitation light was transmitted through a ring light (MA25, Dolan-Jenner) to illuminate sample evenly. The sample of AquaSheet was placed on a piece of 500 μm wafer to remain a low intensity background. The emission images were acquired by an FLIR CCD camera (BFS-U3-04S2C-C, Teledyne FLIR) attached with a lens (f/2.8˜56, 60 mm focal length, Nikon). An Arduino-driven automatic filter wheel (LCFW5, ThorLabs) was placed between the sample and CCD camera, with two band-pass filters (515˜535 nm for AF488 emission channel or 574˜596 nm for AF568 emission channel, respectively, Chroma Technology Corporation); the filter wheel was shifted automatically for acquisition of the AF488 or AF568 emission channels.

Calculation of relative intensity by ratiometric imaging method:

Unlike the reflectance probe system that provides a fully resolved emission spectrum, the imaging system only provides integrated intensities in two spectral bands (515˜535 nm and 574˜596 nm—FIG. 26). To determine the relative intensity, a set of two images were acquired (exposure time=0.5˜4 seconds) for each spectral band, respectively. The first image

$$( {I}_{AF {488}_{ex}}^{AF {488}_{em}} )$$

was captured with AF488 excitation band and detected at AF488 emission band (channel 1); the second image

$$( {I}_{A F {488}_{ex}}^{AF {568}_{em}} )$$

was captured by being excited at AF488 excitation band and collected at AF568 emission band (channel 2). In each pixel, i, of the images, the intensities,

$${I}_{AF {488}_{{ex}_{i}}}^{AF {488}_{em}} and {I}_{AF {488}_{{ex}_{i}}}^{AF {568}_{em}} ,$$

were recorded as grayscale values for pixel-wise evaluation of relative intensities. To perform the background subtraction, the median of a certain area of pristine PDMS with no AquaDust embedded in was considered as the background value, which were

$${Bg}_{AF {488}_{ex}}^{AF {488}_{em}} and {Bg}_{AF {488}_{ex}}^{AF {568}_{em}} ,$$

respectively. By subtracting these two corresponding values, the background-subtracted images are

$${I}_{AF {488}_{{ex}_{i}}}^{′ AF {488}_{em}} and {I}_{AF {488}_{{ex}_{i}}}^{′ AF {568}_{em}} .$$

However, in this imaging method, consideration of bleed-through was necessary because the AF488 emission contributed significantly at the AF568 emission spectral band. This bleed-through contribution (red area under AF488 curve depicted in FIG. 26) needs to be subtracted to evaluate the emission of AF568. Thus, a bleed-through correction factor, a_{c} was defined as the ratio of integrated area of Em_{AF488} in the AF568 emission band

$$( {Area}_{AF 48 {8}_{ex}}^{AF 56 {8}_{em}} ,$$

red area under AF488 emission curve depicted in FIG. 26) to integrated area of Em_{AF488} in the AF488 emission channel

$$( {Area}_{AF {488}_{ex}}^{AF 48 {8}_{em}} ,$$

blue area under AF488 emission curve depicted in FIG. 26):

$${α}_{c} = \frac{{Area}_{AF {488}_{ex}}^{AF {568}_{em}}}{{Area}_{AF {488}_{ex}}^{AF {488}_{em}}} \qquad ( 4 )$$

Here, the calculated bleed-through correction factor, a_{c}≅0.15 from FIG. 26 With this ratio, the corrected, pixel-wise relative intensity (ζ_{corr,i}) was calculated as Eq. 5:

$${ζ}_{corr , i} ( {a}_{w} ) = \frac{{I}_{AF {488}_{{ex}_{i}}}^{′ AF {568}_{em}} - {α}_{c} \times {I}_{AF {488}_{{ex}_{i}}}^{′ AF {488}_{em}}}{{I}_{AF {488}_{{ex}_{i}}}^{′ AF {488}_{em}} + {I}_{AF {488}_{{ex}_{i}}}^{′ AF {568}_{em}} - {α}_{c} \times {I}_{AF {488}_{{ex}_{i}}}^{′ AF {488}_{em}}} \qquad ( 5 )$$

The analysis and determination for ζ_{corr} based on the pixel-wise relative intensity (ζ_{corr,i}) from ratiometric imaging measurements (FIG. 27)

As mentioned in previous section, a set of two images

$${I}_{AF 48 {8}_{ex}}^{AF 48 {8}_{em}}$$

### (FIG. 27(A)) and

$${I}_{AF 48 {8}_{ex}}^{AF 56 {8}_{em}}$$

(FIG. 27(B)) were acquired for analysis. The saturated pixels for either image (Intensity>65300 (a.u.)) were excluded for the following analysis. The pixel-wise relative intensity (ζ_{corr,i}) was calculated through Eq. 5. FIG. 27(C) shows the spatial distribution of ζ_{corr,i} for the corresponding

$${I}_{AF {488}_{{ex}_{i}}}^{′ AF {488}_{em}} and {I}_{AF {488}_{{ex}_{i}}}^{′ AF {568}_{em}} .$$

For one measurement of steady-state characterization, a pixel count distribution of calculated ζ_{corr,i}(a_{w}) can be plotted (FIG. 27(D)), and the median of this distribution determined: ζ_{corr}(a_{w}). The calibration of this pixel-wise measurement of relative intensity was performed by assessing ζ_{corr}(a_{w}) for samples at equilibrium with a series of known, steady water activities, a_{w} in vapor or solution as shown in FIG. 28(B).

$$ζ ( t ) = Δ {ζ}_{0} ( 1 - {e}^{\frac{t}{{τ}_{AqS}}} ) + {ζ}_{initial} \qquad ( 6 )$$

Where t represents the elapse time, ζ(t) is the relative intensity at time t, Δζ₀ represents the amplitude, τ_{AqS} the time constant, and ζᵢₙᵢₜᵢₐₗ refers to the relative intensity when t<0 (i.e., before the step change of water activity). According to this fit, the time constant, τ_{AqS}=537 seconds (red dashed curve in FIG. 21(A)).

This transient is controlled by diffusion of water molecules through the AquaSheet matrix through thickness (z-axis). To test this hypothesis, a prediction for Eq. 6 was derived based on one-dimensional (1-D) transient diffusion with a constant water activity at the top surface and no flux boundary condition at the bottom surface (as shown in FIG. 21(C)). The following form for the time constant is found:

$${τ}_{AqS} = \frac{4 {h}_{p}^{2}}{{π}^{2} D} \qquad ( 7 )$$

where hₚ [m] is the thickness of the AquaSheet and D [m² s⁻¹] is the diffusivity of water in PDMS. With the mechanism proposed in Section. Theory and Design, the water molecules diffuse through the PDMS matrix and then interact locally with AquaDust. As a result, the diffusivity of water molecules is deployed in PDMS to derive the time constant. Previous studies of the transport of water (both liquid and vapor state) in silicone matrix via experiments and simulation reports values, D=0.8 to 4×10⁻⁹ m²/s. With hₚ=0.8 mm, the value of τ_{AqS}=64 (with D=4×10⁻⁹ m²/s) to τ_{AqS}=324 (with D=0.8×10⁻⁹ m²/s) seconds based on Eq. 7.

The discrepancy between the experimentally determined time constant of 537 seconds and the theoretically evaluated range of 64 to 327 seconds can be attributed to several factors based on the assumptions of the model. Firstly, AquaSheet is a composite material consisting of both PDMS and PAAm-based AquaDust. Using the diffusivity of water molecules in PDMS directly may not accurately reflect the true diffusivity within the composite, leading to discrepancies. Additionally, although the transport behavior of water molecules in silicone has been extensively studied, the true mechanism remains unresolved. Factors such as the activity of water, the cluster state of water, and the intrinsic properties of silicone itself can influence diffusivity. Therefore, the diffusion of water in the PDMS-based AquaSheet is likely dependent on a variety of complex factors, contributing to the observed differences between experimental and theoretical characteristic times.

As a sensing patch, AquaSheet is expected to quickly respond to changes in water activity, which means the time constant should be smaller. The theoretical time constant calculated by Eq. 7 can provide guideline on decreasing the time constant by either increase the diffusivity or decrease the thickness of AquaSheet. Since diffusivity is determined by the material properties, reducing the thickness of AquaSheet is necessary to enhance water molecule diffusion. For current design, both of the reflectance fluorescent spectra and images were acquired along with z-axis in FIG. 21(C). This z-axis also served as the main transport path of water molecules, which determined the characteristic time constant. However, the intensity of fluorescence from AquaSheet is determined through the total fluorophores per unit area at x-y plane, which means that the thicker the AquaSheet is, the higher the intensity of AquaSheet will be acquired. This tradeoff could be moderated by increasing the strength of signal with higher concentrations of dyes, longer integration times, more intense source of excitation, or a more sensitive optical detector.

AquaSheet on reporting temporally and spatially resolved water activity:

The water that permeates soils presents a particularly important and challenging context for thermodynamic and dynamic measurements due to the coexistence of both liquid and vapor phases and the structural complexity arising from the distributed grain sizes. This complexity makes it difficult to accurately assess water movement and availability. However, the thermodynamic status of water in soil is crucial as it governs the availability of water to plants and microorganisms, directly impacting their growth and metabolic activities. Also, water in soils and plants controls the terrestrial hydrological cycle via evaporation and transpiration into the atmosphere. With the growing world population and the increasing impacts of climate change, pressures on our water resources are intensifying. This creates an urgent need to better understand the status of water within this hydrological cycle to manage and conserve these vital resources effectively. In geotechnical engineering, water in soil is essential for the transport of moisture and the movement of thermal energy. Within the soil-plant continuum, the thermodynamic availability and dynamic transport of water influence the water use efficiency and the harvest of crops. Moreover, several open questions remain in this field, spanning from macroscopic scales, such as water transport through multilayers of soils with varying properties, to microscopic scales, like water movement across the soil-root-rhizosphere interface. Addressing these questions requires the development of advanced tools to monitor and understand both the thermodynamic and dynamic aspects of water within the plant-soil continuum.

Existing tools for localized, point measurements of water activity (or water potential) in soils have been extensively studied. Among these, thermocouple psychrometry is the most heavily researched technique for in situ hygrometry in soils and plants within a research context. However, high-temperature sensitivity still poses technical challenges for being widely adopted outside of research context. Tensiometers, which operate based on the equilibration of an internal bulk volume of pure liquid water with an unsaturated external vapor phase across a wettable, porous membrane, also face limitations. Capillarity within the membrane facilitates this equilibrium, but tensiometers have large form factors (sensing area>10 cm²), prohibiting high spatial resolution and restricting them to point measurements.^{10,70} There are also existing reporting methods for imaging spatial distribution of water content in soils, which includes neutron radiography, nuclear magnetic resonance, and X-ray. Nevertheless, the reporting of water content cannot reveal the water activity (i.e., water potential), which represents the thermodynamic state of water to participate in geological, physical, chemical, and biological processes in soils.

AquaSheet offers a novel solution by capturing the spatiotemporal distribution of water activity in capillary-driven processes within porous media. Soil, in particular, is one of the most challenging porous media due to its chemical and structural heterogeneity. Moreover, the presence of water in multiple phases (vapor and liquid) and states that are below or near saturation further complicates the measurement of water activity in soils. AquaSheet addresses these challenges, providing a more comprehensive understanding of water dynamics in complex soil environments.

Ratiometric imaging method as described in Section Theory and Design can provide both spatial and temporal information for extracting spatiotemporal water activity. To deploy AquaSheet for reporting water activity of a soil drying process, a calibration process was performed by assessing ζ_{corr}(a_{w}) for AquaSheet at equilibrium with a series of known, steady vapor water activities ranging from 0.576 to 0.985, with three replicates for each steady water activity. FIG. 28(A) shows the images for near saturation (a_{w}˜1, upper row) and undersaturation (0<a_{w}<1, lower row). Both channels exhibit higher intensities at near saturation case and lower intensities at undersaturation case, with varying degrees of decrease in response to changes in water activity. This result show agreement with the spectral data shown in FIG. 20. FIG. 28(B) shows the calibration curve for relative intensity against water activity of AquaSheet using the ratiometric imaging method. The error bars represent the in-replicate standard deviation of the pixel count distribution with respect to the median ζ_{corr}(a_{w}) as illustrated in FIG. 22(D). The wide distribution of relative intensity in this calibration can be attributed to the heterogeneity of AquaDust distribution in AquaSheet. As described previously, the change of fluorescence of AquaSheet results from the combination of FRET and quenching behavior, and the local concentration of AquaDust in AquaSheet can influence the fluorescence in response to water activities. Qualitatively, AquaSheet shows higher sensitivity and lower uncertainty when the water activity is close to saturation.

In FIG. 22, a drying experiment was performed to investigate the evolution of water status in soil by AquaSheet. An optical cell was designed to observe the drying process with AquaSheet as shown in FIG. 22(A-B). With one side open to ambient and the other three sides sealed by an O-ring, this optical cell is aimed to create a one-dimensional progression of drying through the soil sample. The drying direction is defined as x-axis, the direction perpendicular to the drying direction as y-axis and the through-thickness direction as z-axis, which is illustrated in FIG. 29(A).

Measurements:
spatial resolution and spatial “graininess” of images. Magnitude and potential sources of heterogeneity.
uncertainty in water potential (e.g., based on s.d. of measured values across a slice at a given depth). Magnitude and potential origins.
Comment on intrinsic transient in AquaSheet, i.e., dynamics of lateral progression vs. tau for through-thickness equilibration.

FIG. 22(C) presents the time evolution of the spatial distribution of water activity of soil drying process across 45 hours. The spatial resolution is determined by the native resolution of the CCD camera and the setting of the system with the conversion ratio of 0.0069 mm/pixel and region of interest covering an area of 9.7×3.6 mm². The graininess of the spatial distribution of water activity originated from two sources: (i) the heterogeneous distribution of AquaDust in AquaSheet, and (ii) the challenging chemical and structural heterogeneity of soil. Although the optical cell was designed to apply pressure on the soil sample to be more tightly packed, the heterogeneous grain sizes create undulating surface, which leads to uneven emission reflectance background for AquaSheet.

The water activity of each pixel in the spatial distribution is determined through linear interpolation and extrapolation with respect to the calibration points shown in FIG. 28(B). For the calibration process, background subtraction was performed to extract the relationship between relative intensity and water activity. However, when soil serves as the background of system, the native heterogeneity and the high reflectance of dry soil pose challenges for the analysis. In FIG. 22(C), the relative intensity is calculated without performing background subtraction.

Also, in this system, AquaSheet and soil can be considered as one system with multiple paths for water to be transported despite the hydrophobic nature of PDMS. However, as a sensing patch, AquaSheet should only report the water activity through-thickness and not influence the drying process along the drying direction shown in FIG. 22(C). To not influence the water transport in soil, AquaSheet should be with low conductance and low capacitance to water molecules.

In FIG. 22(C), when drying time equals to 0 hours, the AquaSheet is reporting dryer water activity, and this is because AquaSheet was stored in a dry environment prior to the experiment was performed. As a result, there is a time delay for AquaSheet to reach equilibrium of AquaSheet and the wet soil sample. Within five hours, there is no drying front visible but only the balance between AquaSheet and soil, and this can be explained by the capacity of soil to accommodate water in its porous structure. After 5 hours, the soil sample starts to dry with vapor invading into the pores of soil, and the drying front is obvious to be observed. To further understand the progression of drying front, the one-dimension progression of drying front along x-axis is extracted as shown in FIG. 31. This one-dimensional progression file is plotted through extracting the median of all y pixels along each x pixel, and the shaded area represents the standard deviation of the pixel value distribution. The velocity of the drying front retreatment is decreasing due to the low conductivity of water vapor at low water activity. Also, the water activity is pinned at ˜45% relative humidity, which corresponds to the ambient ˜50% relative humidity and a forced convective air through fan. This result on reporting spatiotemporal distribution of soil drying revealed the unprecedented ability to capture the water activity distribution in porous media, enabling direct measurements on water activity (in contrast to conventional tools to measure water content) for the first time.

However, there are still outstanding challenges which remain unresolved for AquaSheet. The temporal resolution is determined by the transient of AquaSheet, which currently is suitable for steady measurements (steady water potential of plants) and slow dynamic processes (such as drying dynamic of porous media). In contrast, AquaSheet may not be able to capture fast dynamic processes such as imbibition or irrigation. As a result, the tradeoff between transient and intensity should be further investigated. For in situ applications of AquaSheet, there are also several open questions that remain unresolved. AquaSheet relies on direct intact with this interface. Consequently, the compatibility between AquaSheet and systems of interest needed to be studied prior for the applications. In addition, the optical transparent windows of the conjugated dyes on AquaDust, the background fluorescence from targeted system, and PDMS matrix are important to be investigated for minimizing the influence on each other. Also, taking the advantage of PDMS, AquaSheet also can be potentially fabricated into complex geometries for different applications including: (i) coupled with optical fiber for optical implants on measuring water status in plants, (ii) curved interfaces in rhizotron imaging tube for soil-root interface, (iii) 3-D printed AquaSheet into 3D structures for more tortuous and complicated structures. With the ability to report water activity for either spatially averaged, temporally resolved or spatiotemporal distribution, AquaSheet shows the potential to be applied for continuous water activity measurement in plants, tracking water and energy transport in porous median, in operando tracking of water in fuel cells, and integration into processing systems and packing materials for foods and pharmaceuticals.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. An optical sensor patch for measuring water activity, the optical sensor patch comprising:
a sheet configured to be in equilibrium with water activity in an environment of the optical sensor patch;
a pair of distinct fluorescent dyes dispersed within the sheet, wherein a fluorescence spectra of the optical sensor patch is dependent on the water activity in the environment of the optical sensor patch.

2. The optical sensor patch of claim 1, wherein the sheet is a polydimethylsiloxane (PDMS) matrix.

3. The optical sensor patch of claim 1, wherein the pair of distinct fluorescent dyes are covalently linked in a polymer matrix of a hydrogel nanoparticle dispersed in the sheet

4. The optical sensing patch of claim 3, wherein changes in the fluorescence spectra of the optical sensing patch are based on changes in self-quenching of the pair of distinct fluorescent dyes and changes in Förster Resonance Energy Transfer (FRET) between the pair of distinct fluorescent dyes.

5. The optical sensor patch of claim 1, wherein the pair of distinct fluorescent dyes are freely dispersed in the sheet.

6. The optical sensor patch of claim 5, wherein changes in the fluorescence spectra of the optical sensor patch are based on changes in self-quenching of the pair of distinct fluorescent dyes.

7. The optical sensor patch of claim 1, wherein the optical sensor patch is configured to be implanted in a plant to perform in vivo measurements of changes in the fluorescence spectra of the optic sensor patch based on changes in the water activity in the in vivo environment of the plant.

8. An optical implant comprising:
a first window in a substrate configured to receive the optical sensor patch of claim 1;
a first groove in the substrate configured to receive a first optical fiber to direct light to and receive light from the optical sensor patch within the first window; and
a tip located proximate the first window, the tip configured for insertion of at least a portion of the optical implant into an environment.

9. The optical implant of claim 8, wherein the substrate is formed of polymethyl methacrylate (PMMA).

10. The optical implant of claim 8, wherein the substrate has a thickness of about 0.8 mm.

11. The optical implant of any claim 8, wherein the substrate further comprises:
a second window configured to receive a second optical patch therein; and
a second groove configured to receive a second optical fiber to direct light to and receive light from the second optical sensor patch within the second window.

12. The optical implant of claim 11, wherein the second window is located proximate to the tip.

13. The optical implant of claim 11, wherein the second optical patch serves as a reference.

14. The optical implant of claim 8, wherein the substrate further comprises a reflective coating.

15. A system for measuring water activity, the system comprising:
the optical sensor patch of claim 1;
a fiber optic cable;
a light source coupled to the fiber optic cable to provide light directed at the optical sensor patch; and
a measurement device coupled to the fiber optic cable to receive and analyze modulated light from the patch.

16. The system of claim 15, wherein a distal portion of the fiber optic cable is embedded within the optical sensor patch.

17. The system of claim 15, wherein therein the fiber optic cable is a 200 μm optical.

18. A method of measuring water activity, the method comprising;
locating the optical sensor patch of claim 1 in an environment;
measuring changes in the fluorescence spectra of the optical sensor patch; and
determining the water activity in the environment of the optical sensor patch based on the changes in the fluorescence spectra.

19. The method of claim 18, wherein the optical sensor patch is provided in an optical implant, the method further comprising:
implanting the optical implant into a plant to measure the in vivo water activity of the plant.

20. The method of claim 19. wherein the water activity is correlated with one or more analytes or physical parameters of the plant over a period of time.
